(19) United States
(12) Patent Application Publication
(10) Pub. No.: US 2007/0232677 A1
(43) Pub. Date: Oct. 4, 2007

(54) Treatment and prevention of vascular hyperplasia using polyamine and polyamine analog compounds

(76) Inventors: Laurence J. Marton, Palo Alto, CA (US); Randolph M. Johnson, Half Moon Bay, CA (US)

Correspondence Address: MORRISON & FOERSTER LLP, 755 PAGE MILL RD, PALO ALTO, CA, 94304-1018, US

(21) Appl. No.: 11/724,521

(22) Filed: Mar. 14, 2007

Related U.S. Application Data
(60) Provisional application No. 60/782,612, filed on Mar. 14, 2006.

(51) Int. Cl. A61K 31/409; A61K 31/13

(52) U.S. Cl. 514/410; 514/579; 514/671; 514/674

(57) ABSTRACT

This disclosure relates to methods of inhibiting vascular hyperplasia using polyamines, polyamine analogs, and conformationally restricted polyamine analogs, or a conjugate of a polyamine, polyamine analog, or conformationally restricted polyamine analog. Polyamines, polyamine analogs, conformationally restricted polyamine analogs, and conjugates thereof are useful in reducing stenosis and restenosis of blood vessels and grafts.

### CROSS-REFERENCE TO RELATED APPLICATIONS

This application claims priority benefit of U.S. Provisional Application No. 60/782,612, filed Mar. 14, 2006. The entire content of that application is hereby incorporated by reference herein.

### TECHNICAL FIELD

This application relates to methods of inhibiting vascular and intimal hyperplasia, including stenosis and restenosis of vasculature, using polyamine analogs, particularly conformationally restricted polyamine analogs.

### BACKGROUND

The vascular system in the human body plays a critical role. Oxygen, nutrients, and other vital substances are carried to tissues by the vascular system, while carbon dioxide and other waste products are removed. Interruption of the blood flow to a part of the body causes significant damage to that tissue; when the blood flow to the heart or brain is interrupted, severe disability or death can result.

Human blood vessels are composed of an inner lining of endothelial cells, called the tunica intima; a middle portion of concentrically-arranged smooth muscle cells called the tunica media; and an outer layer of thick, longitudinally-arranged connective tissue called the tunica adventitia. A variety of problems involving the abnormal growth of vascular cells can arise in these layers, particularly the intima, both due to pathology and to medical intervention to relieve pathology.

Arteriosclerotic and atherosclerotic lesions are a common occurrence in populations in industrialized nations, and play a central role in the mortality associated with vascular disease in developed countries. As lesions progress in severity and atherosclerotic plaque accumulates, narrowing of blood vessel diameter and loss of elasticity of the artery occurs, resulting in reduced blood flow to the tissues supplied by the affected vessel. Eventually an ischemic event can occur, such as a heart attack or stroke.

In order to prevent such a devastating event, various medical treatments are in common use. Percutaneous transluminal coronary angioplasty (PTCA), also referred to as balloon angioplasty, is often used to re-open an artery narrowed by atherosclerotic plaque. In balloon angioplasty, a catheter is threaded inside a blood vessel to the location of the plaque, followed by inflation of a small balloon, which compresses the plaque against the arterial wall. This procedure, while effective for opening the artery, damages the tunica intima. Subsequently, vascular hyperplasia—a condition marked by abnormal, unusual, or excessive growth of cells of the vascular tissue—can occur, as cells in the intima proliferate in response to the damage. Intimal hyperplasia often progresses to the point where the blood vessel is blocked again; this process is known as restenosis.

Other vascular interventions can also result in hyperplasia and stenosis. Atherectomy and endarterectomy are procedures for removing plaque from the walls of blood vessels, after which vascular hyperplasia may occur. Vascular hyperplasia is also a complication that can result after formation of vascular fistulas, or implantation of vascular device grafts, for frequent vascular access. (Frequent vascular access is required, for example, by dialysis patients.)

The abnormal, unusual, or excessive growth of cells of the vascular tissue that occurs in vascular hyperplasia can cause serious circulatory problems, including cardiac ischemia, cerebral ischemia, or claudication. Accordingly, there is a need for therapeutic interventions to treat or prevent vascular hyperplasia, as well as other disorders of the vascular system involving abnormal cell growth of the vasculature, such as hypertrophy of vascular cells.

### DISCLOSURE OF THE INVENTION

The present invention relates to methods of treating and/or preventing abnormal vascular cell growth and/or abnormal vascular cell proliferation with polyamines and polyamine analogs, such as non-conformationally restricted polyamine analogs or conformationally restricted polyamine analogs; or with conjugates of polyamines, polyamine analogs, non-conformationally restricted polyamine analogs, or conformationally restricted polyamine analogs, such as conjugates with peptides, amino acids, and porphyrin compounds. In one embodiment, the present invention relates to methods of treating and/or preventing vascular hyperplasia with polyamines and polyamine analogs, such as non-conformationally restricted polyamine analogs or conformationally restricted polyamine analogs. In another embodiment, the present invention relates to methods of treating and/or preventing vascular hyperplasia with non-conformationally restricted polyamine analogs or conformationally restricted polyamine analogs conjugated to porphyrin molecules, such as mesoporphyrin IX. In another embodiment, the present invention relates to methods of treating and/or preventing vascular hyperplasia with non-conformationally restricted polyamine analogs or conformationally restricted polyamine analogs conjugated to peptides. In another embodiment, the present invention relates to methods of treating and/or preventing vascular hyperplasia with non-conformationally restricted polyamine analogs or conformationally restricted polyamine analogs conjugated to amino acids. The methods of the invention embrace the use of polyamines, polyamine analogs, non-conformationally restricted polyamine analogs, or conformationally restricted polyamine analogs; conjugates of polyamines, polyamine analogs, non-conformationally restricted polyamine analogs, or conformationally restricted polyamine analogs; and compositions (including pharmaceutical compositions) comprising a polyamine, polyamine analog, non-conformationally restricted polyamine analogs, or conformationally restricted polyamine analog or conjugates thereof for treating and/or preventing vascular hyperplasia. In one embodiment, the treatment and/or prevention of vascular hyperplasia occurs subsequent to balloon angioplasty. In another embodiment, the treatment and/or prevention of vascular hyperplasia occurs subsequent to placement of a stent. In another embodiment, the treatment and/or prevention of vascular hyperplasia occurs subsequent to creation or placement of a vascular graft. In one embodiment, the polyamine analog(s) is conformationally restricted. In another embodiment, the vascular hyperplasia excludes cancer or other malignancies.

In another embodiment, the present invention excludes atherosclerosis-related conditions, such as atherosclerotic lesions, atherosclerotic plaque, or atheromas from the diseases to be treated or prevented. In another embodiment, the present invention includes atherosclerosis-related conditions, such as atherosclerotic lesions, atherosclerotic plaque, or atheromas, as one of the diseases to be treated or prevented by the methods described herein only if such atherosclerosis-related condition has been previously treated by a mechanical or surgical intervention.

In one embodiment, the one or more compounds for treating and/or preventing abnormal vascular cell growth and/or abnormal vascular cell proliferation is one or more conformationally restricted polyamine analogs. In one embodiment, the only conformational restriction of the polyamine analog is due to a carbon—carbon double bond (an ethenyl group, C═C) in the molecule; in this embodiment, a proviso is added to any or all of the embodiments below that the only conformational restriction of the polyamine analog is due to a carbon—carbon double bond. In another embodiment, the only conformational restriction of the polyamine analog is due to a cycloalkyl group, such as a cyclopropyl group, in the molecule; in this embodiment, a proviso is added to any or all of the embodiments below that the only conformational restriction of the polyamine analog is due to a cycloalkyl group, or due to a cyclopropyl group.

In one embodiment, the conformationally restricted polyamine analog is selected from among compounds of the formula:
E—NH—B—A—B—NH—B—A—B—NH—B—A—B—NH—B—A—B—NH—E
where A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of: a single bond, C₁-C₆ alkyl, and C₂-C₆ alkenyl; and E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 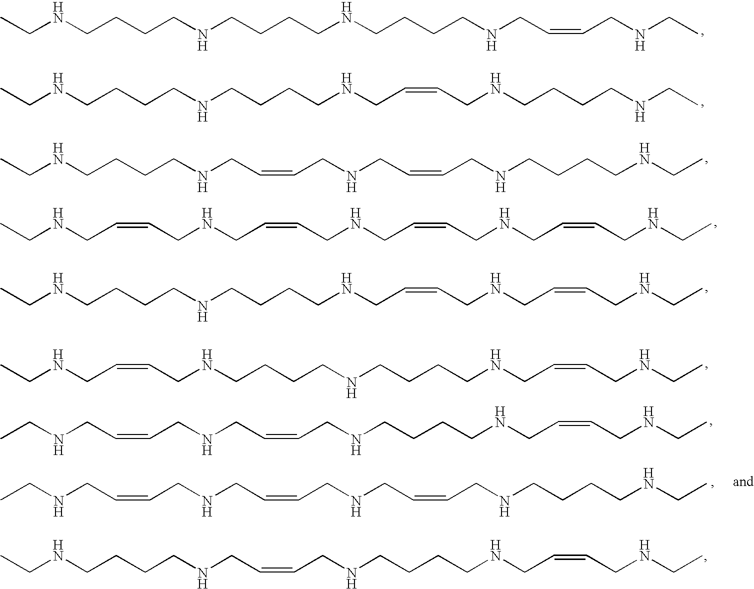
and all salts, hydrates, solvates, and stereoisomers thereof.

In another embodiment, the conformationally restricted polyamine analog is selected from among the group of compounds of the formula:
E—NH—B—A—B—NH—B—A—B—NH—B—A—B—NH(—B—A—B—NH)ₓ—E
wherein A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; and x is an integer from 2 to 16; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. In another embodiment, x is 4, 6, 8, or 10. In another embodiment, x is 4. In another embodiment, x is 6. In another embodiment, x is 8. In another embodiment, x is 10. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 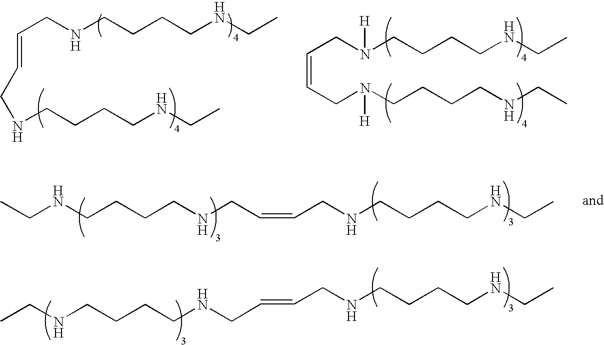
and all salts, hydrates, solvates, and stereoisomers thereof.

In another embodiment, the conformationally restricted polyamine analog is selected from among the group of compounds of the formula
E—NH—B—A—B—NH—B—A—B—NH—B—A—B—NH(—B—A—B—NH)ₓ—E
wherein A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of C₁-C₆ alkyl, C₁-C₆ alkanol, C₃-C₆ cycloalkanol, and C₃-C₆ hydroxyaryl, with the proviso that at least one E moiety be selected from the group consisting of C₁-C₆ alkanol, C₃-C₆ cycloalkanol, and C₃-C₆ hydroxyaryl; and x is an integer from 0 to 16; and all salts, hydrates, solvates, and stereoisomers thereof. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 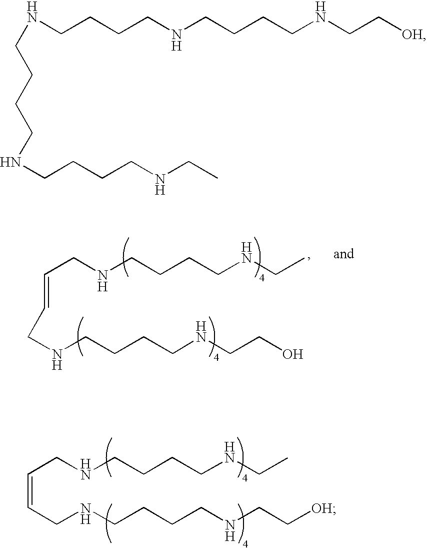
and all salts, hydrates, solvates, and stereoisomers thereof.

In another embodiment, the conformationally restricted polyamine analog is selected from among the group of compounds of the formula
E—NH—D—NH—B—A—B—NH—D—NH—E
wherein A is independently selected from the group consisting of C₂-C₆ alkene and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl; and all salts, hydrates, solvates, and stereoisomers thereof. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 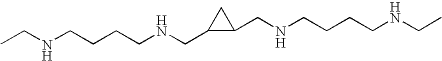
(CGC-11093, formerly SL-11093), 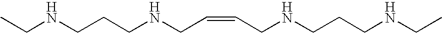
(CGC-11047, formerly SL-11047); 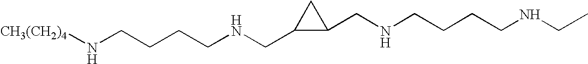
(CGC-11302, formerly SL-11302),
and all salts, hydrates, solvates, and stereoisomers thereof.

In another embodiment, the conformnationally restricted polyamine analog is selected from macrocyclic polyamines of the formula: 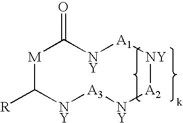
where A₁, each A₂ (if present), and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In additional embodiments, the Y group is —H or —CH₃. In another embodiment, A₁, each A₂ (if present), and A₃ are independently selected from C₂-C₄ alkyl. In yet another embodiment, M is —CH₂—.

In another embodiment, the conformationally restricted polyamine analog is selected from macrocyclic polyamine analogs of the formula 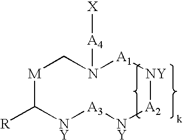
where A₁, each A₂ (if present), and A₃ are independently selected from C₁-C₈ alkyl; where A₄is selected from C₁-C₈ alkyl or a nonentity; where X is selected from —H, —Z, —CN, —NH₂, —C(═O)—C₁-C₈ alkyl, or —NHZ, with the proviso that when A₄is a nonentity, X is —H, —C(═O)—C₁-C₈ alkyl, or —Z; where Z is selected from the group consisting of an amino protecting group, an amino capping group, an amino acid, and a peptide; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In certain embodiments, A₄is a nonentity. In other embodiments, X is —Z, and —Z is —H. In other embodiments, X is —Z, and —Z is 4-morpholinocarbonyl. In other embodiments, X is —Z and —Z is acetyl. In other embodiments, X is —Z and —Z is t-Boc or Fmoc. In other embodiments, Y is —CH₃. In other embodiments, M is —CH₂—. In still further embodiments, k is 1. In further embodiments, A₁ and A₃ are —CH₂CH₂CH₂—. In still further embodiments, —CH₂CH₂CH₂CH₂—. In still further embodiments, R is —C₁₃H₂₇. In yet further embodiments, one or more of the specific limitations on A₄, X, Z, Y, M, k, A₁, A₃, and R are combined.

In further embodiments of these macrocyclic polyamine analog compounds, A₄ is C₁-C₈ alkyl, X is —NHZ, and Z is selected from one of the 20 genetically encoded amino acids (alanine, cysteine, aspartic acid, glutamic acid, phenylalanine, glycine, histidine, isoleucine, lysine, methionine, asparagine, proline, glutamine, arginine, serine, threonine, valine, tryptophan, tyrosine), a peptide of the formula acetyl-SKLQL—, a peptide of the formula acetyl-SKLQ-beta-alanine—, or a peptide of the formula acetyl-SKLQ—. In these cases, where Z is an amino acid or peptide, the therapeutic agent to be used is a polyamine-amino acid conjugate or polyamine-peptide conjugate.

In another embodiment, the conformationally restricted polyamine analog is CGC-11047. In another embodiment, the conformationally restricted polyamine analog is CGC-11093. In another embodiment, the conformationally restricted polyamine analog is CGC-11144. In another embodiment, the conformationally restricted polyamine analog is CGC-11150. In another embodiment, the conformationally restricted polyamine analog is CGC-11157. In another embodiment, the conformationally restricted polyamine analog is CGC-11158. In another embodiment, the conformationally restricted polyamine analog is CGC-11302. In another embodiment, the conjugate of a conformationally restricted polyamine analog with a porphyrin compound is CGC-11217. In another embodiment, the conjugate of a conformationally restricted polyamine analog with a porphyrin compound is CGC-11237. In other embodiments, any salt, solvate, hydrate, stereoisomer, or polymorph of CGC-11047, CGC-11093, CGC-11144, CGC-11150, CGC-11157, CGC-11158, CGC-11302, CGC-11217, or CGC-11237 can be used for treatment or prevention of vascular hyperplasia.

In one embodiment, the one or more compounds for treating and/or preventing abnormal vascular cell growth and/or abnormal vascular cell proliferation is one or more non-conformationally restricted polyamine analogs. In another embodiment, the invention embraces use of compounds for treating or preventing vascular hyperplasia, where the compounds are non-conformationally restricted polyamines of the formula: 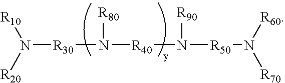
where R₁₀, R₂₀, R₆₀, and R₇₀ are independently selected from H, methyl, ethyl, n-propyl, isopropyl, n-butyl, isobutyl, sec-butyl, and t-butyl;
where each R₈₀ and R₉₀ are independently selected from H, methyl, ethyl, n-propyl, isopropyl, n-butyl, isobutyl, sec-butyl, and t-butyl;
where R₃₀, each R₄₀, and R₅₀ are independently selected from:
—CH₂CH₂CH₂CH₂—
—CHOHCH₂CH₂CH₂—
—CH₂CHOHCH₂CH₂—
—CH₂CH₂CHOHCH₂—
—CH₂CH₂CH₂CHOH—
—CH₂CH₂CH₂—
—CHOHCH₂CH₂—
—CH₂CHOHCH₂—
—CH₂CH₂CHOH;—
and where y is an integer selected from 5, 6, 7, 8, 9, 10, 11, 12, and 13; and all salts thereof.

In one embodiment, at least one R₃₀, R₄₀, or R₅₀ is independently selected from:
—CHOHCH₂CH₂CH₂—
—CH₂CHOHCH₂CH₂—
—CH₂CH₂CHOHCH₂—
—CH₂CH₂CH₂CHOH—
—CHOHCH₂CH₂—
—CH₂CHOHCH₂— and
—CH₂CH₂CHOH—.

In another embodiment, at least one R₃₀, R₄₀, or R₅₀ is independently selected from:
—CHOHCH₂CH₂CH₂—
—CH₂CHOHCH₂CH₂—
—CH₂CH₂CHOHCH₂—; and
—CH₂CH₂CH₂CHOH—.

In another embodiment, at least one of R₃₀ and R₅₀ is independently selected from:
—CHOHCH₂CH₂CH₂—
—CH₂CHOHCH₂CH₂—
—CH₂CH₂CHOHCH₂—; and
—CH₂CH₂CH₂CHOH—.

In another embodiment, each R₄₀ is independently selected from the group consisting of —CH₂CH₂CH₂— and —CH₂CH₂CH₂CH₂—. In another embodiment, R₃₀ is independently selected from the group consisting of —CH₂CH₂CH₂— and —CH₂CH₂CH₂CH₂—. In another embodiment, R₅₀ is independently selected from the group consisting of —CH₂CH₂CH₂— and —CH₂CH₂CH₂CH₂—. In another embodiment, R₃₀ and R₅₀ are independently selected from the group consisting of —CH₂CH₂CH₂— and —CH₂CH₂CH₂CH₂—. In another embodiment, R₄₀ and R₅₀ are independently selected from the group consisting of —CH₂CH₂CH₂— and —CH₂CH₂CH₂CH₂—. In another embodiment, R₃₀ and R₄₀ are independently selected from the group consisting of —CH₂CH₂CH₂— and —CH₂CH₂CH₂CH₂—. In another embodiment, R₃₀, each R₄₀, and R₅₀ are independently selected from the group consisting of —CH₂CH₂CH₂— and —CH₂CH₂CH₂CH₂—. In one embodiment of the foregoing embodiments, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In another embodiment, each R₄₀ is —CH₂CH₂CH₂CH₂—. In another embodiment, R₃₀ is —CH₂CH₂CH₂CH₂—. In another embodiment, R₅₀ is —CH₂CH₂CH₂CH₂—. In another embodiment, R₃₀ and R₅₀ are —CH₂CH₂CH₂CH₂—. In another embodiment, each R₄₀ and R₅₀ are —CH₂CH₂CH₂CH₂—. In another embodiment, R₃₀ and each R₄₀ are —CH₂CH₂CH₂CH₂—. In another embodiment, R₃₀, each R₄₀, and R₅₀ are —CH₂CH₂CH₂CH₂—. In one embodiment of the foregoing embodiments, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In another embodiment, each R₄₀ is —CH₂CH₂CH₂—. In another embodiment, R₃₀ is —CH₂CH₂CH₂—. In another embodiment, R₅₀ is —CH₂CH₂CH₂—. In another embodiment, R₃₀ and R₅₀ are —CH₂CH₂CH₂—. In another embodiment, each R₄₀ and R₅₀ are —CH₂CH₂CH₂—. In another embodiment, R₃₀ and each R₄₀ are —CH₂CH₂CH₂—. In another embodiment, R₃₀, each R₄₀, and R₅₀ are —CH₂CH₂CH₂—. In one embodiment of the foregoing embodiments, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In one embodiment, R₁₀, R₂₀, R₆₀, and R₇₀ are independently selected from H, methyl, ethyl, n-propyl, and n-butyl. In another embodiment, each R₈₀ and R₉₀ are independently selected from H, methyl, ethyl, n-propyl, and n-butyl. In another embodiment, R₁₀, R₂₀, R₆₀, and R₇₀ are independently selected from H, methyl, ethyl, n-propyl, and n-butyl, and each R₈₀ and R₉₀ are independently selected from H, methyl, ethyl, n-propyl, and n-butyl.

In another embodiment, R₉₀ and each R₈₀ are H. In another embodiment, R₁₀ is H, R₂₀ is ethyl, R₆₀ is H, and R₇₀ is ethyl. In one embodiment of the foregoing embodiments, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In another embodiment, R₃₀, each R_{40,} and R₅₀ are —CH₂CH₂CH₂CH₂—, and R₉₀ and each R₈₀ are H. In another embodiment, R₃₀, each R_{40,} and R₅₀ are —CH₂CH₂CH₂CH₂—, and R₁₀ is H, R₂₀ is ethyl, R₆₀ is H, and R₇₀ is ethyl. In another embodiment, R₃₀, each R_{40,} and R₅₀ are —CH₂CH₂CH₂CH₂—, R₉₀ and each R₈₀ are H, and R₁₀ is H, R₂₀ is ethyl, R₆₀ is H, and R₇₀ is ethyl. In one embodiment of the foregoing embodiments, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In another embodiment, the invention embraces use of compounds for treating or preventing vascular hyperplasia, where the compounds are non-conformationally restricted polyamines of the formula:
CH₃CH₂NHCH₂CH₂CH₂CH₂(NHCH₂CH₂CH₂CH₂)_{y}NHCH₂CH₂CH₂CH₂NHCH₂CH₃
where y=5, 6, 7, 8, 9, 10, 11, 12, or 13. In yet another embodiment, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11. In any of the foregoing embodiments, any salt of any compound, or the non-salt free base of any compound can be used.

In another embodiment, the invention embraces use of compounds for treating or preventing vascular hyperplasia, where the compounds are non-conformationally restricted polyamines of the formula:
CH₃CH₂NHCH₂CH₂CH₂(NHCH₂CH₂CH₂)_{y}NHCH₂CH₂CH₂NHCH₂CH₃
where y=5, 6, 7, 8, 9, 10, 11, 12, or 13. In yet another embodiment, y=5, 7, 9, 11, or 13. In yet another embodiment, y=6, 8, 10, or 12. In yet another embodiment, y=5, 7, 9, or 11. In yet another embodiment, y=5. In yet another embodiment, y=7. In yet another embodiment, y=9. In yet another embodiment, y=11.

In another embodiment, the invention embraces use of compounds for treating or preventing vascular hyperplasia, where the compounds are non-conformationally restricted polyamines of the formula:
CH₃CH₂—NH—(CH₂CH₂CH₂CH₂—NH—)₉—CH₂CH₃ (CGC-11159),
CH₃CH₂—NH—(CH₂CH₂CH₂CH₂—NH—)₇—CH₂CH₃ (CGC-11160),
CH₃CH₂—NH—(CH₂CH₂CH₂CH₂—NH—)₁₃—CH₂CH₃ (CGC-11175),
CH₃CH₂—NH—(CH₂CH₂CH₂CH₂—NH—)₁₁—CH₂CH₃ (CGC-11226),
and all salts thereof.

In another embodiment, the invention embraces a method of treating and/or preventing vascular hyperplasia, comprising administering one or more polyamine analogs in a therapeutically effective amount to a subject in need of treatment and/or prevention of vascular hyperplasia. The polyamine analog can be a conformationally restricted polyamine analog. The polyamine analog can be a non-conformationally restricted polyamine analog. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule. In other embodiments, the vascular hyperplasia can be subsequent to balloon angioplasty, stent placement, creation of a vascular graft, implantation of a vascular graft device, atherectomy, or endarterectomy. In other embodiments, the vascular hyperplasia can be subsequent to pathology which arises prior to intervention. The method embraces administration of a polyamine or polyamine analog, which can be a conformationally restricted polyamine analog or a non-conformationally restricted polyamine analog, in an amount sufficient to effect any one or more of the following: reduce vascular hyperplasia; suppress vascular hyperplasia; or delay the development of vascular hyperplasia. The invention also embraces administration of a polyamine, polyamine analog, non-conformationally restricted polyamine analog, or conformationally restricted polyamine analog, or conjugates thereof in an amount sufficient to cause regression of vascular hyperplasia. The invention also embraces administration of a polyamine, polyamine analog, non-conformationally restricted polyamine analog, or conformationally restricted polyamine analog, or conjugates thereof in an amount sufficient to prevent vascular hyperplasia, for example, to prevent restenosis after angioplasty, to prevent restenosis after stent placement, to prevent stenosis of a vascular graft, to prevent stenosis of a vascular graft device, to prevent restenosis after atherectomy, or to prevent restenosis after endarterectomy. The reduction, suppression, delay, regression, or prevention of vascular hyperplasia can be partially complete, substantially complete, or complete. In another embodiment, the non-conformationally restricted polyamine analog is CGC-11159. In another embodiment, the conformationally restricted polyamine analog is CGC-11047. In another embodiment, the conformationally restricted polyamine analog is CGC-11093. In another embodiment, the conformationally restricted polyamine analog is CGC-11144. In another embodiment, the conformationally restricted polyamine analog is CGC-11150. In another embodiment, the conformationally restricted polyamine analog is CGC-11157. In another embodiment, the conformationally restricted polyamine analog is CGC-11158. In another embodiment, the conformationally restricted polyamine analog is CGC-11302. In another embodiment, the conjugate of a conformationally restricted polyamine analog with a porphyrin compound is CGC-11217. In another embodiment, the conjugate of a conformationally restricted polyamine analog with a porphyrin compound is CGC-11237.

In another embodiment, the polyamine, polyamine analog, non-conformationally restricted polyamine analog, or conformationally restricted polyamine analog, or conjugates thereof is administered as a preventive or prophylactic measure. In one embodiment, the only conformational restriction of the polyamine analog is due to a carbon—carbon double bond (an ethenyl group, C═C) in the molecule. In another embodiment, the only conformational restriction of the polyamine analog is due to a cycloalkyl group, such as a cyclopropyl group, in the molecule. The polyamine analog or conformationally restricted polyamine analog can be administered to patients at risk of vascular hyperplasia, including vascular hyperplasia causing stenosis or restenosis after angioplasty and/or stent placement, or vascular hyperplasia causing stenosis of a vascular graft, at varying intervals and via various methods of administration. Patients at risk of vascular hyperplasia include patients with vascular grafts used for dialysis, patients who have undergone angioplasty, and patients who have had stents implanted in a blood vessel.

In one embodiment, the polyamine, polyamine analog, non-conformationally restricted polyamine analog, or conformationally restricted polyamine analog, or conjugate thereof is present in a formulation suitable for intravenous or intraarterial administration, where the formulation comprises a polyamine, polyamine analog, or conformationally restricted polyamine analog or conjugate thereof and a pharmaceutical carrier suitable for intravenous or intraarterial administration. In another embodiment, the polyamine, polyamine analog, non-conformationally restricted polyamine analog, or conformationally restricted polyamine analog or conjugate thereof is present in a formulation suitable for administration in or near the tunica adventitia (periadventitial administration), where the formulation comprises a polyamine, polyamine analog, or conformationally restricted polyamine analog or conjugate thereof and a pharmaceutical carrier suitable for administration in or near the tunica adventitia (periadventitial administration). In another embodiment, the polyamine, polyamine analog, non-conformationally restricted polyamine analog, or conformationally restricted polyamine analog or conjugate thereof is present in a formulation suitable for injection in or near the tunica adventitia (periadventitial injection), where the formulation comprises a polyamine, polyamine analog, or conformationally restricted polyamine analog or conjugate thereof and a pharmaceutical carrier suitable for injection in or near the tunica adventitia (enriadventitial injection). The intravenous or intraarterial formulations can be administered by various routes, such as injection into an artery or vein from outside the body, release into an artery or vein from a catheter at or near a site at risk of vascular hyperplasia, or injection in or near the tunica adventitia (periadventitial injection) at or near a site at risk of vascular hyperplasia using a catheter.

In one embodiment, the invention embraces administration of a polyamine, polyamine analog, non-conformationally restricted polyamine analog, or conformationally restricted polyamine analog or conjugate thereof or conjugate thereof about once a week for about two to about twelve months. In another embodiment, the invention embraces administration of a polyamine, polyamine analog, non-conformationally restricted polyamine analog, or conformationally restricted polyamine analog or conjugate thereof about once every two weeks for about two to about twelve months. In another embodiment, the invention embraces administration of a polyamine, polyamine analog, non-conformationally restricted polyamine analog, or conformationally restricted polyamine analog or conjugate thereof about once every three weeks for about two to about twelve months. In another embodiment, the aforementioned administration regimens comprise intravenous or intraarterial administration of the polyamine, polyamine analog, non-conformationally restricted polyamine analog, or conformationally restricted polyamine analog or conjugate thereof. In another embodiment, the aforementioned administration regimens comprise periadventitial injection of the polyamine, polyamine analog, non-conformationally restricted polyamine analog, or conformationally restricted polyamine analog or conjugate thereof. In another embodiment, the aforementioned administration regimens comprise administration of CGC-11159. In another embodiment, the aforementioned administration regimens comprise administration of CGC-11047. In another embodiment, the aforementioned administration regimens comprise administration of CGC-11093. In another embodiment, the aforementioned administration regimens comprise administration of CGC-11144. In another embodiment, the aforementioned administration regimens comprise administration of CGC-11150. In another embodiment, the aforementioned administration regimens comprise administration of CGC-11157. In another embodiment, the aforementioned administration regimens comprise administration of CGC-11158. In another embodiment, the aforementioned administration regimens comprise administration of CGC-11302. In another embodiment, the aforementioned administration regimens comprise administration of CGC-11217. In another embodiment, the aforementioned administration regimens comprise administration of CGC-11237.

In another embodiment, the invention embraces administration of a polyamine, polyamine analog, non-conformationally restricted polyamine analog, or conformationally restricted polyamine analog or conjugate thereof in a sustained release formulation. In one embodiment, the sustained release formulation is implanted in or near a site at risk of vascular hyperplasia. In another embodiment, the sustained release formulation is coated onto a device for implantation, such as a stent or vascular graft device.

In another embodiment, the invention embraces a polyamine, polyamine analog, non-conformationally restricted polyamine analog, or conformationally restricted polyamine analog or conjugate thereof in a sustained release formulation, comprising a polyamine, polyamine analog, or conformationally restricted polyamine analog or conjugate thereof in a sustained release formulation or sustained release device. In another embodiment, the invention embraces a polyamine, polyamine analog, non-conformationally restricted polyamine analog, or conformationally restricted polyamine analog or conjugate thereof in a sustained release formulation suitable for administration or implantation in or near a site in a blood vessel at risk of vascular hyperplasia, comprising a polyamine, polyamine analog, non-conformationally restricted polyamine analog, or conformationally restricted polyamine analog or conjugate thereof in a sustained release formulation or sustained release device suitable for administration or implantation in or near the tunica adventitia (periadventitial administration or implantation). In another embodiment, the invention embraces a polyamine, polyamine analog, non-conformationally restricted polyamine analog, or conformationally restricted polyamine analog or conjugate thereof in a sustained release formulation or sustained release device suitable for administration or implantation in the periadventitial tissue, comprising a polyamine analog, a non-conformationally restricted polyamine analog, or a conformationally restricted polyamine analog in a sustained release formulation or sustained release device suitable for administration or implantation in the periadventitial tissue.

In another embodiment, the invention embraces the use of a polyamine, polyamine analog, non-conformationally restricted polyamine analog, or conformationally restricted polyamine analog or conjugate thereof in a sustained release formulation, comprising formulating a polyamine, polyamine analog, non-conformationally restricted polyamine analog, or conformationally restricted polyamine analog or conjugate thereof in a sustained release formulation or sustained release device, and administering or implanting the formulation or device in or near a site in a blood vessel at risk of vascular hyperplasia. In one embodiment, the administering or implanting of the sustained release formulation or sustained release device comprises administering or implanting the formulation or device in or near the tunica adventitia (periadventitial administration or implantation).

### DETAILED DESCRIPTION OF THE INVENTION

A “subject” or a “patient” refers to a vertebrate, preferably a mammal, more preferably a human. The polyamine analogs described herein or incorporated by reference herein are used to treat vertebrates, preferably mammals, more preferably humans.

“Treating” or “to treat” a disease using the methods of the invention is defined as administering one or more polyamines, polyamine analogs, or conformationally restricted polyamine analogs, or one or more conjugates thereof (or a composition comprising those compounds), with or without additional therapeutic agents, in order to palliate, ameliorate, stabilize, reverse, slow, delay, reduce, or eliminate either the disease or the symptoms of the disease, or to retard or stop the progression of the disease or of symptoms of the disease. To “prevent” a disease means to suppress the occurrence of a disease or symptoms of a disease before its clinical manifestation. Prevention or suppression can be partial or total. “Therapeutic use” of the polyamines, polyamine analogs, conformationally restricted polyamine analogs, and conjugates thereof is defined as using one or more of those compounds (or a composition comprising those compounds) to treat or to prevent a disease, as defined above. An “effective amount,” “amount effective [to],” or “therapeutically effective amount” is an amount sufficient to treat or prevent a disease, as defined above.

By “undesirable cell proliferation” is meant any condition where cells are growing or multiplying, and such growth or multiplication is undesirable (for example, causing disease and/or unwanted symptoms, or having potential to cause disease and/or unwanted symptoms). In one embodiment, benign tumors are excluded from conditions characterized by undesirable cell proliferation. In another embodiment, malignant (cancerous) tumors are excluded from conditions characterized by undesirable cell proliferation. In another embodiment, both benign and malignant (cancerous) tumors are excluded from conditions characterized by undesirable cell proliferation.

By “abnormal vascular cell growth” is meant any type of cell growth in the vascular system which can cause pathology. Abnormal vascular cell growth includes vascular hyperplasia, which is an undesirable proliferation of vascular cells. Vascular hyperplasia includes undesirable cell growth in any portion of the vascular system, such as the tunica adventitia, tunica media, and tunica intima, and includes all types of abnormally growing cells, such as hyperplasia involving endothelial cells, fibroblasts, or smooth muscle cells. Vascular hyperplasia of the tunica intima is also referred to as intimal hyperplasia or neointimal hyperplasia. Abnormal vascular cell growth also includes hypertrophy of cells of the vascular system, such as the development of foam cells. Abnormal vascular cell growth also includes abnormal changes in cell structure or organization. In one embodiment, benign tumors are excluded from conditions characterized by abnormal vascular cell growth. In another embodiment, malignant (cancerous) tumors are excluded from conditions characterized by abnormal vascular cell growth. In another embodiment, both benign and malignant (cancerous) tumors are excluded from conditions characterized by abnormal vascular cell growth.

By a “polyamine,” a term well-understood in the art, is meant any of a group of naturally occurring, saturated aliphatic, straight-chain amines. Examples of polyamines are spermine, spermidine, and putrescine. Natural polyamines are typically derived biosynthetically from amino acids; polyamines are reviewed in Marton et al. (1995) Ann. Rev. Pharm. Toxicol. 35:55-91. By “polyamine analog” is meant an organic cation structurally similar but non-identical to naturally occurring polyamines, that is, a polyamine analog is a non-naturally-occurring, saturated or unsaturated aliphatic amine. Polyamine analogs can be branched or un-branched. Polyamine analogs include, but are not limited to, BE-4444 [1,19-bis (ethylamino)-5,10,15-triazanonadecane]; BE-333 [N1,N11 -diethylnorspermine; DENSPM; 1,11 -bis (ethylamino)-4,8-diazaundecane; thermine; Warner-Parke-Davis]; BE-33 [N1,N7-bis(ethyl) norspermidine]; BE-34 [N1,N8-bis(ethyl) spermidine]; BE-44 [N1,N9-bis(ethyl) homospermidine]; BE-343 [N1,N12-bis(ethyl) spermine; diethylspermine-N1-N12; DESPM]; BE-373 [N,N′-bis (3-ethylamino) propyl)-1,7-heptane diamine, Merrell-Dow]; BE-444 [N1,N14-bis(ethyl) homospermine; diethylhomospermine-N1-N14]; BE-3443 [1,17-bis(ethylamino)-4,9,14-triazaheptadecane]; and BE-4334 [1,17-bis(ethylamino)-5,9,13-triazaheptadecane]; 1,12-Me₂-SPM [1,12-dimethylspermine]. See also Feuerstein et al. (1991); Gosule et al. (1978)J. Mol. Biol. 121:311-326; Behe et al. (1981) Proc. Natl. Acad. Sci. USA 78:1619-23; Jain et al. (1989) Biochem. 28:2360-2364; Basu et al. (1990) Biochem. J. 269:329-334; Porter et al. (1988), Advances in Enzyme Regulation, Pergamon Press, pp. 57-79; Frydman et al. (1992) Proc. Natl. Acad. Sci. USA 89:9186-9191; and Fernandez et al. (1994) Cell Mol. Biol. 40: 933-944.

By “conformationally restricted polyamine analog” is meant that, in a polyamine analog, at least two amino groups in the molecule are locked or limited in spatial configuration relative to each other. The amino groups within the molecule may be primary, secondary, tertiary, or quartenary, and are preferably primary or secondary amino groups, more preferably secondary amino groups. The relative movement of two amino groups can be restricted, for example, by incorporation of a cyclic or unsaturated moiety between them (exemplified, but not limited to, a ring, such as a three-carbon ring, four-carbon ring, five-carbon-ring, six-carbon ring, or a double or triple bond, such as a double or triple carbon bond). Polyamines can also be constrained by incorporation of two or more amino groups into a macrocyclic structure. Groups restricting conformational flexibility by means of steric hindrance, yet favorable to the therapeutic effects of the compound, can also be used. A conformationally restricted polyamine analog can comprise at least two amino groups which are conformationally restricted relative to each other; a conformationally restricted polyamine analog can also further comprise amino groups which are not conformationally restricted relative to other amino groups. Flexible molecules such as spermine and BE-444 can have a myriad of conformations and are therefore not conformationally restricted. Conformationally restricted polyamine analogs include, but are not limited to, the compounds disclosed in International Patent Application WO 98/17624, U.S. Pat. No. 5,889,061, and U.S. Pat. No. 6,392,098; the compounds disclosed in WO 00/66587 and U.S. Pat. No. 6,794,545; and the compounds disclosed in U.S. Pat. No. 6,982,351, United States Patent Application Publication Nos. 2003/0072715, 2003/0195377, and International Patent Applications WO 02/10142, and WO 03/050072. Several of these compounds are depicted below in Table 1. All of the polyamine analog compounds (both conformationally restricted polyamine analog compounds and non-conformationally restricted polyamine analog compounds) disclosed in those patents or patent applications, including but not limited to the specification, claims, tables, examples, figures, and schemes of those patents or patent applications, are expressly incorporated by reference herein as compounds useful in the invention. The conformationally restricted polyamine analog compounds disclosed in those patents or patent applications, including but not limited to the specification, claims, tables, examples, figures, and schemes of those patents or patent applications, are expressly incorporated by reference herein as compounds useful in the invention.

In certain embodiments, the saturated oligoamines (which are non-conformationally restricted polyamine analogs) disclosed in U.S. Patent Application Publication No. 2003/0130356 can be used for treatment and/or prevention of vascular hyperplasia, and all oligoamine compounds disclosed therein, including but not limited to the specification, claims, tables, examples, figures, and schemes of that patent application, are expressly incorporated by reference herein as compounds useful in the invention.

In certain additional embodiments, the polyamine analog-peptide conjugates disclosed in U.S. Pat. No. 6,649,587 can be used for treatment and/or prevention of vascular hyperplasia, and all polyamine analog-peptide conjugates disclosed therein, including but not limited to the specification, claims, tables, examples, figures, and schemes of that patent, are expressly incorporated by reference herein as compounds useful in the invention.

In certain additional embodiments, the polyamine analog-amino acid conjugates disclosed in International Patent Application WO 02/38105 can be used for treatment and/or prevention of vascular hyperplasia, and all polyamine analog-amino acid conjugates disclosed therein, including but not limited to the specification, claims, tables, examples, figures, and schemes of that patent application, are expressly incorporated by reference herein as compounds useful in the invention.

In certain additional embodiments, the polyamine analog-porphyrin conjugates disclosed in WO 00/66587, U.S. Pat. No. 6,794,545, International Patent Application WO 04/002991 and United States Patent Application Publication No. 2004/0152687 can be used for treatment and/or prevention of vascular hyperplasia, and all polyamine analog-porphyrin conjugates disclosed therein, including but not limited to the specification, claims, tables, examples, figures, and schemes of those patent publications, are expressly incorporated by reference herein as compounds useful in the invention. Two such compounds are SL-11217:

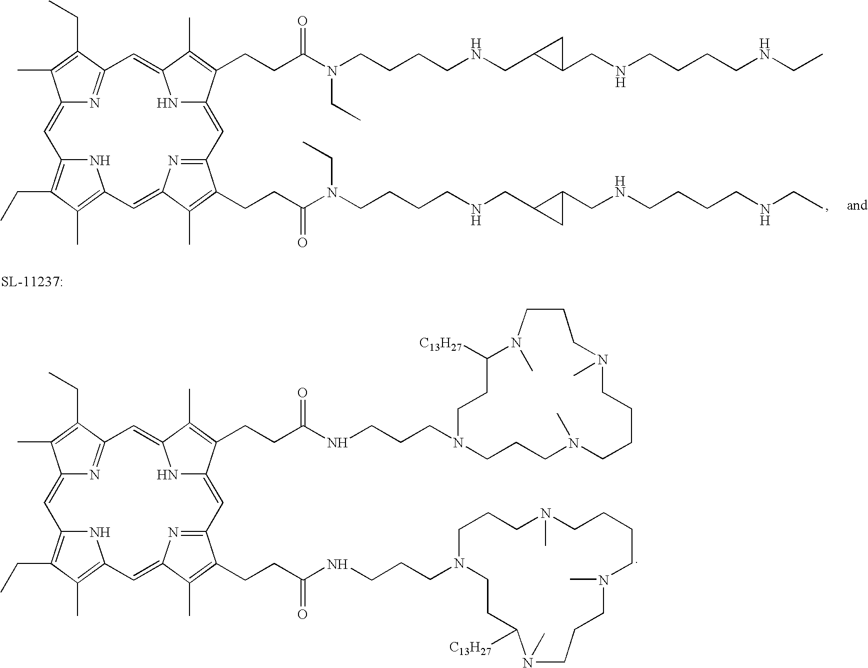

One preferred subset of polyamine analogs and conformationally restricted polyamine analogs are those containing 8, 10, 12, or 14 nitrogen atoms. Such compounds include CGC-11144 and CC-11150 (also known as SL-11144 and SL-11150, respectively), each of which contains 10 nitrogens, and CGC-11157 and CGC-11158 (also known as SL-11157 and SL-11158, respectively), each of which contains 8 nitrogens. Another preferred subset of polyamine analogs and conformationally restricted analogs comprises the compounds known as CGC-11093 and CGC-11047 (also known as SL-11093 and SL-11047, respectively), each of which contains 4 nitrogens. Another preferred conformationally restricted analog is the compound CGC-11302 (also known as SL-11302).

One preferred subset of non-conformationaly restricted polyamine analogs are the compounds CGC-11159, CGC-11160, CGC-11175, and CGC-11226, particularly CGC-11159.

The invention includes the use of all of the compounds described herein or incorporated by reference herein, including any and all stereoisomers, salts, hydrates and solvates of the compounds described herein or incorporated by reference herein. The invention also includes the use of all compounds described herein or incorporated by reference herein in their non-salt (free base) form. The invention also includes the use of all compounds described herein or incorporated by reference herein in their non-salt, non-hydrate/non-solvate form. Particularly preferred are pharmaceutically acceptable salts. Pharmaceutically acceptable salts are those salts which retain the biological activity of the free bases and which are not biologically or otherwise undesirable. The desired salt may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of the compounds with amino acids, such as aspartate salts and glutamate salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

The invention also includes all crystalline and non-crystalline forms of the compounds, including all polymorphs of the compounds.

The term “alkyl” refers to saturated aliphatic groups including straight-chain, branched-chain, cyclic groups, and combinations thereof, having the number of carbon atoms specified, or if no number is specified, having up to 12 carbon atoms, with preferred subsets of alkyl groups including C₁-C₁₂, C₁-C₁₀, C₁-C₈, C₁-C₆, and C₁-C₄ alkyl groups. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cyclic groups can consist of one ring, including, but not limited to, groups such as cycloheptyl, or multiple fused rings, including, but not limited to, groups such as adamantyl or norbomyl.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups.

“Hydroxyalkyl” specifically refers to alkyl groups having the number of carbon atoms specified substituted with one —OH group. Thus, “C₃ linear hydroxyalkyl” refers to —CH₂CH₂CHOH—, —CH₂CHOHCH₂—, and —CHOHCH₂CH₂—.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH=CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or multiple condensed rings (including, but not limited to, groups such as naphthyl or anthryl), and includes both unsubstituted and substituted aryl groups. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—,1-ethyl-6-propylpiperidino, 2-ethylthiophenyl, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, thiophene, or furyl) or multiple condensed rings (including, but not limited to, examples such as imidazolyl, indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) as well as the carbon atoms of the group can be substituted. The heteroatom(s) can also be in oxidized form, if chemically possible.

The term “alkylaryl” refers to an alkyl group having the number of carbon atoms designated, appended to one, two, or three aryl groups.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, and t-butoxy.

The term “alkanoate” as used herein refers to an ionized carboxylic acid group, such as acetate (CH₃C(═O)—O⁽⁻¹⁾), propionate (CH₃CH₂C(═O)—O⁽⁻¹⁾), and the like. “Alkyl alkanoate” refers to a carboxylic acid esterified with an alkoxy group, such as ethyl acetate (CH₃C(═O)—O—CH₂CH₃). “ω-haloalkyl alkanoate” refers to an alkyl alkanoate bearing a halogen atom on the alkanoate carbon atom furthest from the carboxyl group; thus, ethyl ω-bromo propionate refers to ethyl 3-bromopropionate, methyl ω-chloro n-butanoate refers to methyl 4-chloro n-butanoate, etc.

The terms “halo” and “halogen” as used herein refer to Cl, Br, F or I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Amino protecting groups include, but are not limited to, mesitylenesulfonyl (Mes), benzyloxycarbonyl (CBz or Z), t-butyloxycarbonyl (Boc), t-butyldimethylsilyl (TBDIMS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Hydroxyl protecting groups include, but are not limited to, Fmoc, TBDIMS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

Examples of compounds useful in the invention are depicted in Table 1. While some of the compounds are depicted as salts, such as the hydrochloride salt, it is to be understood that the disclosure in the table embraces all salts, hydrates, and solvates of the compounds depicted therein, as well as the non-salt, non-hydrate/non-solvate form of the compound, as is well understood by the skilled artisan. Where stereochemistry is not expressly indicated, the disclosure of the compounds is intended to embrace all possible stereoisomers, including the pure isomers, racemic mixtures, and mixtures of isomers in any proportion. Table 1 includes both non-conformationally restricted polyamine analogs and conformationally restricted polyamine analogs. Both types of polyamine analogs are useful in the invention.

TABLE 1

| Compound | Structure |
|---|---|
| CGC-11027 (formerly SL-11027) | 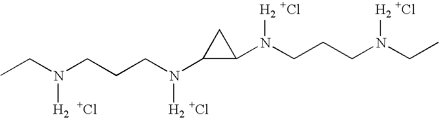 |
| CGC-11028 (formerly SL-11028) | 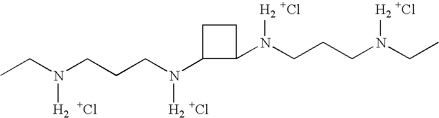 |
| CGC-11029 (formerly SL-11029) | 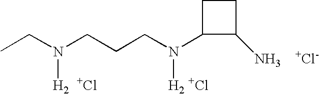 |
| CGC-11033 (formerly SL-11033) | 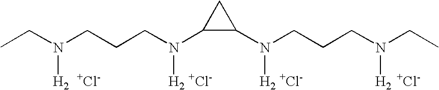 |
| CGC-11034 (formerly SL-11034) | 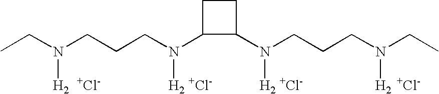 |
| CGC-11035 (formerly SL-11035) | 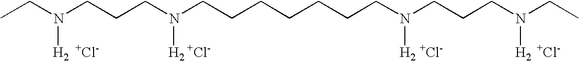 |
| CGC-11036 (formerly SL-11036) | 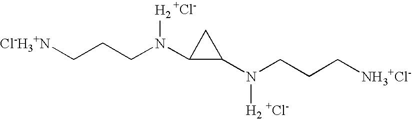 |
| CGC-11037 (formerly SL-11037) | 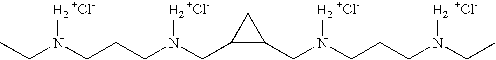 |
| CGC-11038 (formerly SL-11038) | 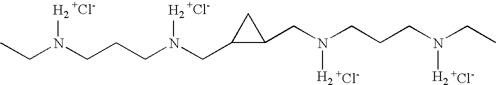 |
| CGC-11043 (formerly SL-11043) | 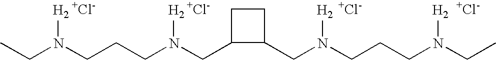 |
| CGC-11044 (formerly SL-11044) | 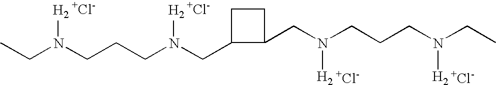 |
| CGC-11047 (formerly SL-11047) | 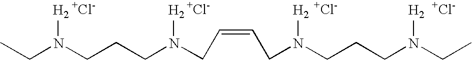 |
| CGC-11048 (formerly SL-11048) | 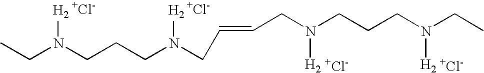 |
| CGC-11050 (formerly SL-11050) | 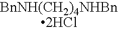 |
| CGC-11061 (formerly SL-11061) | 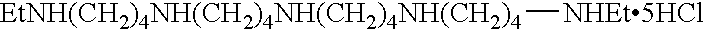 |
| CGC-11093 (formerly SL-11093) | 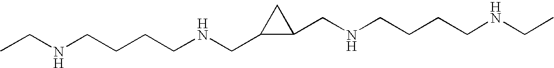 |
| CGC-11094 (formerly SL-11094) | 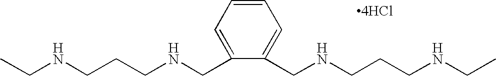 |
| CGC-11098 (formerly SL-11098) | 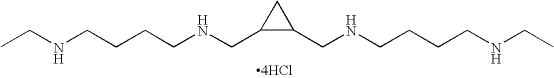 |
| CGC-11099 (formerly SL-11099) | 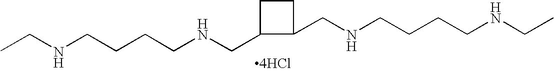 |
| CGC-11100 (formerly SL-11100) | 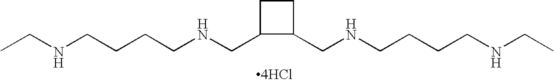 |
| CGC-11101 (formerly SL-11101) | 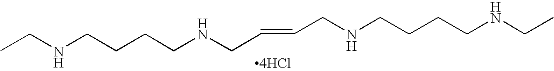 |
| CGC-11102 (formerly SL-11102) | 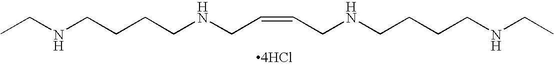 |
| CGC-11103 (formerly SL-11103) | 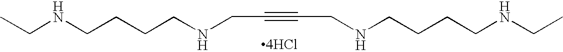 |
| CGC-11104 (formerly SL-11104) | 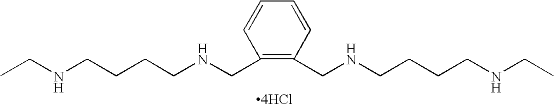 |
| CGC-11105 (formerly SL-11105) | 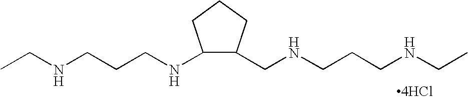 |
| CGC-11108 (formerly SL-11108) | 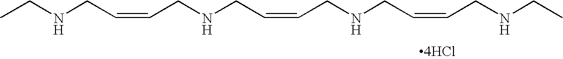 |
| CGC-11114 (formerly SL-11114) | 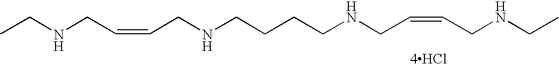 |
| CGC-11119 (formerly SL-11119) | 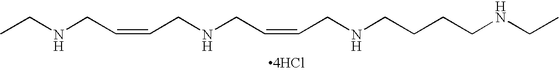 |
| CGC-11090 (formerly SL-11090) | 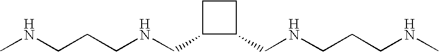 |
| CGC-11091 (formerly SL-11091) | 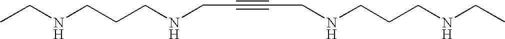 |
| CGC-11092 (formerly SL-11092) | 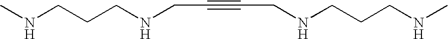 |
| CGC-11101 (formerly SL-11101) | 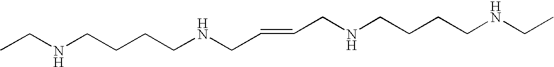 |
| CGC-11103 (formerly SL-11103) | 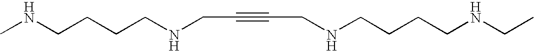 |
| CGC-11114 (formerly SL-11114) | 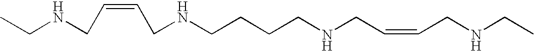 |
| CGC-11118 (formerly SL-11118) | 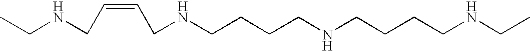 |
| CGC-11121 (formerly SL-11121) | 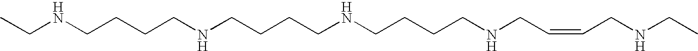 |
| CGC-11122 (formerly SL-11122) | 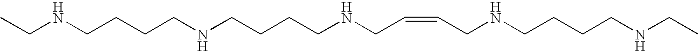 |
| CGC-11123 (formerly SL-11123) | 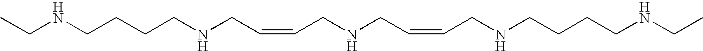 |
| CGC-11124 (formerly SL-11124) | 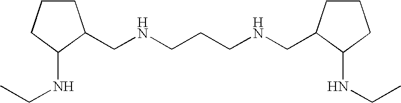 |
| CGC-11126 (formerly SL-11126) | 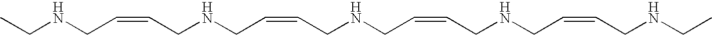 |
| CGC-11127 (formerly SL-11127) | 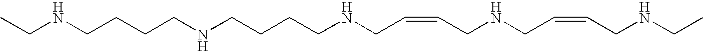 |
| CGC-11128 (formerly SL-11128) | 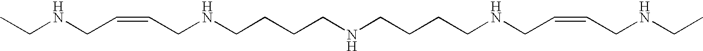 |
| CGC-11129 (formerly SL-11129) | 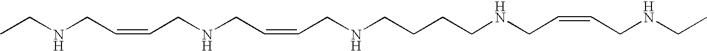 |
| CGC-11130 (formerly SL-11130) | 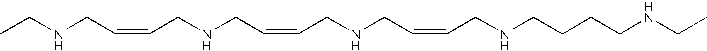 |
| CGC-11132 (formerly SL-11132) | 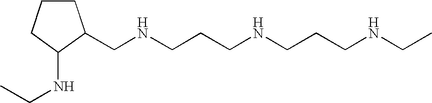 |
| CGC-11133 (formerly SL-11133) | 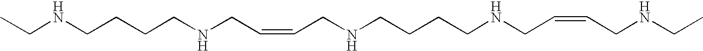 |
| CGC-11134 (formerly SL-11134) | 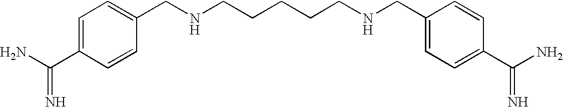 |
| CGC-11135 (formerly SL-11135) | 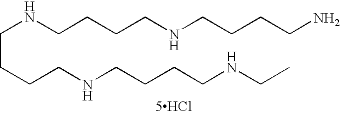 |
| CGC-11136 (formerly SL-11136) | 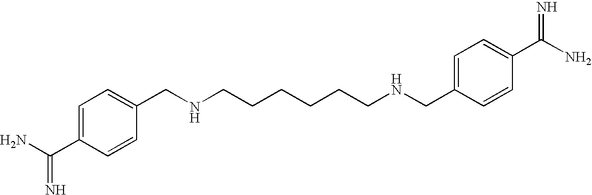 |
| CGC-11137 (formerly SL-11137) | 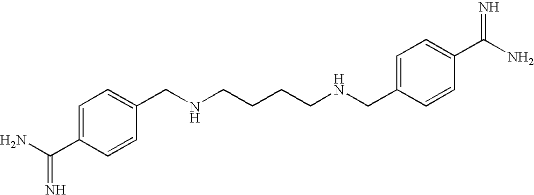 |
| CGC-11141 (formerly SL-11141) | 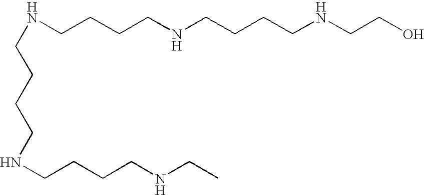 |
| CGC-11143 (formerly SL-11143) | 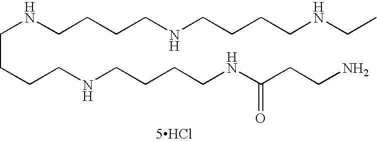 |
| CGC-11144 (formerly SL-11144) | 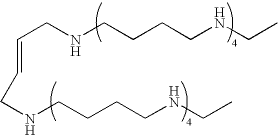 |
| CGC-11150 (formerly SL-11150) | 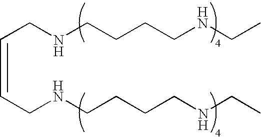 |
| CGC-11155 (formerly SL-11155) | 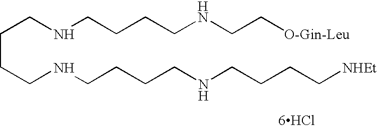 |
| CGC-11157 (formerly SL-11157) | 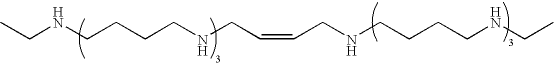 |
| CGC-11158 (formerly SL-11158) | 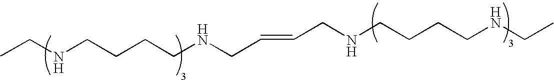 |
| CGC-11201 (formerly SL-11201) | 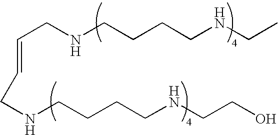 |
| CGC-11202 (formerly SL-11202) | 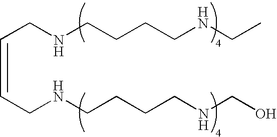 |
| CGC-11174 (formerly SL-11174) | 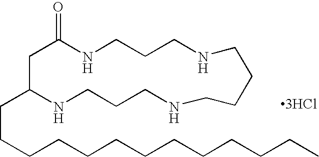 |
| CGC-11197 (formerly SL-11197) | 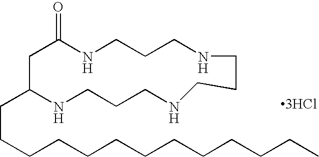 |
| CGC-11199 (formerly SL-11199) | 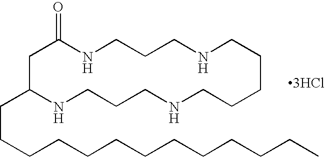 |
| CGC-11200 (formerly SL-11200) | 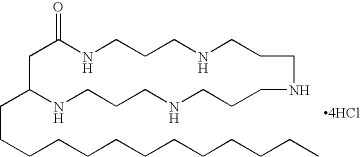 |
| CGC-11208 (formerly SL-11208) | 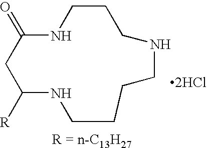 |
| CGC-11217 (formerly SL-11217) | 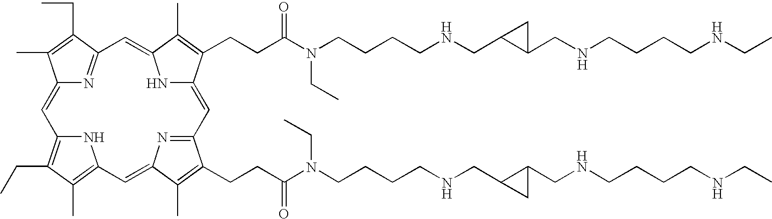 |
| CGC-11237 (formerly SL-11237) | 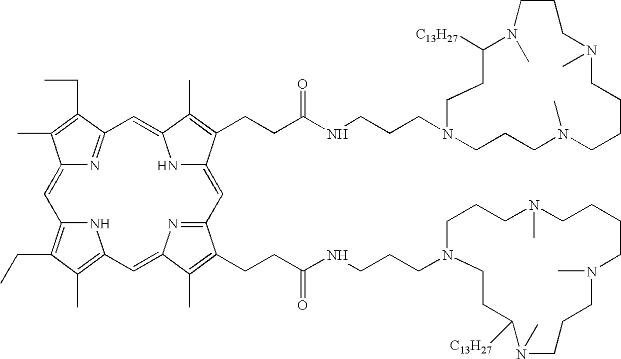 |
| CGC-11238 (formerly SL-11238) | 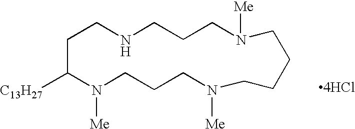 |
| CGC-11239 (formerly SL-11239) | 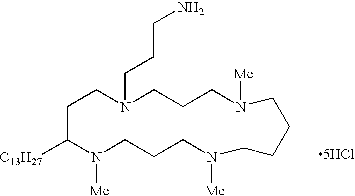 |
| CGC- 11302 (formerly SL-11302) | 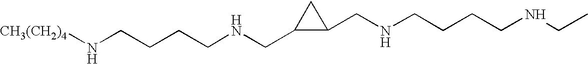 |
| CGC-11159 (formerly- SL-11159) | 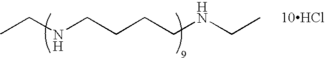 |
| CGC-11160 (formerly SL-11160) | 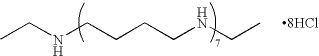 |
| CGC-11175 (formerly SL-11175) | 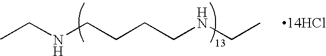 |
| CGC-11226 (formerly SL-11226) | 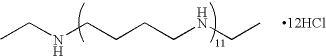 |

Vascular Hyperplasia

The invention embraces methods of treating and/or preventing vascular hyperplasia in a patient or subject with vascular hyperplasia or at risk of developing vascular hyperplasia. Patients or subjects with vascular hyperplasia or at risk of developing vascular hyperplasia have conditions conducive to vascular hyperplasia. These conditions include trauma to a blood vessel, including trauma to the tunica intima; such trauma can be iatrogenic, as when angioplasty is used to open a blocked or partially blocked blood vessel. These conditions also include restenosis of a stent placed in a blood vessel, such as when a stent is placed in a blood vessel subsequently to angioplasty to maintain patency of the blood vessel.

These conditions also include stenosis or restenosis of a vascular graft, such as a vascular graft device used for vascular access, or an arteriovenous graft used for vascular access. When frequent access to the vascular system of a patient or subject is needed (e.g., for dialysis in a patient with renal disease), repeated ordinary venipuncture is impractical. Thus, such patients may undergo an arteriovenous graft, where an artery is connected directly to a vein, bypassing the capillary bed. The increased blood pressure thickens the wall of the vein, making it suitable for repeated venipuncture. Alternatively, a device may be used as a graft, such as a small tube made of a synthetic material that functions as an artificial vein and which can be used to access the vascular system.

These conditions also include use of a blood vessel as a bypass graft during bypass surgery. During bypass surgery, a portion of a blood vessel is taken from part of a patient's body, and used to route blood around (i.e., bypass) a partially or totally blocked portion of another blood vessel. For example, bypass grafts are frequently performed on the coronary arteries, using portions of the internal mammary artery, the saphenous vein, or the radial artery, in order to improve blood supply to the heart. Patients who have undergone bypass surgery are at risk of, and may develop, vascular hyperplasia.

Modes of Administration

Compounds useful in the methods of the invention can be administered to a patient or subject (preferably a human patient or subject) via any route known in the art, including, but not limited to, those disclosed herein. Methods of administration include, but are not limited to, systemic, transpleural, intravenous, oral, intraarterial, intramuscular, topical, via inhalation (e.g. as mists or sprays), via nasal mucosa, subcutaneous, transdermal, intraperitoneal, gastrointestinal, and directly to the wall of a blood vessel or the tissue surrounding a blood vessel. The compounds described or incorporated by reference for use herein can be administered in the form of tablets, pills, powder mixtures, capsules, granules, injectables, creams, solutions, suppositories, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

A preferred route of administration is to the lumen of a blood vessel, to the tunica adventitia of a blood vessel, or to the periadventitial tissue of a blood vessel. The compounds can be administered by intravenous or intraarterial injection. The compounds can also be administered via release from a catheter at or near a site at risk of vascular hyperplasia. Catheters suitable for site-specific administration are well-known in the art; examples are described in US Published Patent Application No. 2002/0095133, International Patent Application No. WO 00/74750, and U.S. Pat. Nos. 5,954,706, and 7,008,411. The compounds can be administered to the subject or patient via an implantable or external pump. Example 2 below demonstrates administration of polyamines via an implanted pump. The compounds can also be administered via injection into or near the tunica adventitia (periadventitial administration) from a catheter at or near a site at risk. Catheters suitable for injection into the periadventitial tissue are also known in the art; examples are described in U.S. Pat. Nos. 6,997,903, 6,319,230, 6,302,870; in US Published Patent Application Nos. 2003/0171734, 2004/0186435, and 2004/0010309; an example of such a catheter which has received FDA premarketing approval is the Mercator MicroSyringe Infusion Catheter. Example 1 below demonstrates administration of polyamines to the periadventitial tissue via use of the Mercator MicroSyringe. The compounds can also be administered to the subject or patient as an implant in the periadventitial tissue. Preferred implants are biocompatible and/or biodegradable sustained release formulations which gradually release the compounds over a period of time. The compounds can also be administered to the subject or patient using iontophoresis.

The pharmaceutical dosage form which contains the compounds for use in the invention is conveniently admixed with a non-toxic pharmaceutical organic carrier or a non-toxic pharmaceutical inorganic carrier, in a unit dosage form containing an amount of compound which is effective for treating or preventing a disease or condition described herein. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the compound(s) to retain its pharmacological activity in the body. Formulations for parenteral and nonparenteral drug delivery are known in the art and are set forth in Remington: The Science and Practice of Pharmacy, 20th Edition, Lippincott, Williams & Wilkins (2000). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers such as sterile water, sterile saline, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the compounds for use in the invention can be administered as injectable dosages of a solution or suspension of the compound in a physiologically acceptable diluent or sterile liquid carrier such as water, saline, or oil, with or without additional surfactants or adjuvants. An illustrative list of carrier oils would include animal and vegetable oils (e.g., peanut oil, soy bean oil), petroleum-derived oils (e.g., mineral oil), and synthetic oils.

For injectable unit doses, sterile liquids such as water, saline, aqueous dextrose and related sugar solutions are preferred liquid carriers. For administration to the periadventitial tissue, the compounds are formulated as a composition suitable for periadventitial administration according to methods known in the art.

The compounds of the present invention can be administered as solutions, suspensions, or emulsions (dispersions) in a suitable intravenous or intraarterial formulation. An appropriate buffer system (e.g., sodium phosphate, sodium acetate, sodium citrate, or sodium borate) may be added. Physiologically balanced solutions of the compounds may be used when the compositions are administered. As used herein, the term “physiologically balanced solution” means a solution which is adapted to maintain the physical structure and function of tissues when introduced into the body. This type of solution will typically contain electrolytes, such as sodium, potassium, calcium, magnesium and/or chloride; an energy source, such as dextrose; and a buffer to maintain the pH of the solution at or near physiological levels. Various solutions of this type are known (e.g., Lactated Ringers Solution).

The compounds of the present invention can be administered with pharmaceutically acceptable preservatives, surfactants, viscosity enhancers, buffers, sodium chloride and water to form aqueous sterile solutions and suspensions.

The pharmaceutical unit dosage chosen is preferably fabricated and administered to provide a therapeutically effective concentration of drug either in the blood, or in tissues of the tunica adventitia or tissues associated with the tunica adventitia, or in tissues of the tunica media or tissues associated with the tunica media, or in tissues of the tunica intima or tissues associated with the tunica intima. The optimal effective concentration of the compounds of the invention can be determined empirically and will depend on the type and severity of the disease, route of administration, disease progression and health, mass and body area of the patient. Such determinations are within the skill of one in the art. Examples of dosages which can be used for systemic administration include, but are not limited to, an effective amount within the dosage range of about 0.1 μg/kg to about 300 mg/kg, or within about 1.0 μg/kg to about 40 mg/kg body weight, or within about 10 μg/kg to about 20 mg/kg body weight, or within about 0.1 mg/kg to about 20 mg/kg body weight, or within about 1 mg/kg to about 20 mg/kg body weight, or within about 0.1 mg/kg to about 10 mg/kg body weight, or within about within about 1 mg/kg to about 10 mg/kg body weight, or within about 0.1 μg/kg to about 10 mg/kg body weight. Examples of dosages which can be used for systemic administration when based on body surface area (expressed in square meters, or m²) include, but are not limited to, an effective amount within the dosage range of about 0.1 μg/m² to about 300 mg/m² body surface area, or within about 10 μg/m² to about 300 mg/m² body surface area, or within about 100 μg/m² to about 300 mg/m² body surface area, or within about 1 mg/m² to about 300 mg/m² body surface area, or within about 10 mg/m² to about 300 mg/m² body surface area, or within about 10 mg/m² to about 200 mg/m² body surface area, or within about 10 mg/m² to about 120 mg/m² body surface area, or within about 40 mg/m² to about 120 mg/m² body surface area, or within about 60 mg/m² to about 100 mg/m² body surface area. For periadventitial administration or injection, examples of dosages which can be used include, but are not limited to, about any of 10 ng, 50 ng, 100 ng, 500 ng, 1 μg, 5 μg, 10 μg, 15 μg, 20 μg, 25 μg, 30 μg, 50 μg, 75 μg, 100 μg, 200 μg, 300 μg, 400 μg, 500 μg, 600 μg, 700 μg, 800 μg, 900 μg, 1 mg, 2 mg, 3 mg, 4 mg, or 5 mg. The dosages may be administered in a single daily dose, or the total daily dosage may be administered in divided dosage of two, three or four times daily. Dosages may also be administered less frequently than daily, for example, six times a week, five times a week, four times a week, three times a week, twice a week, about once a week, about once every two weeks, about once every three weeks, about once every four weeks, about once every six weeks, about once every two months, about once every three months, about once every four months, or about once every six months. For continuous infusion, the drugs can be administered at a concentration between about 0.01 mM to about 100 mM, or between about 0.1 mM to about 50 mM, or about 1 mM to about 20 mM, or about 5 mM to about 15 mM, or about 10 mM, in amounts ranging from about 0.1 ul/hour to about 100 ul/hour, or about 0.1 ul/hour to about 50 ul/hour, or about 0.5 ul/hour to about 25 ul/hour, or about 0.5 ul/hour to about 10 ul/hour, or about 1 ul/hour to about 10 ul/hour, or about 1 ul/hour to about 5 ul/hour, or about 2 ul/hour to about 4 ul/hour, or about 2.5 ul/hour. Infusion can be performed indefinitely, or for about 1 day, or about one week, or about 10 days, or about 4 weeks, or about 2 months, or about 3 months, or about 4 months, or about 6 months, or about 9 months, or about 1 year, or about 2 years, or about 3 years, or about 5 years, or about 10 years. Concentrations, dosages, and flow rates of drug, as well as the specific drug or drugs administered, may be adjusted over time.

In one embodiment, the invention embraces administration of a polyamine, polyamine analog, non-conformationally restricted polyamine analog, or conformationally restricted polyamine analog or conjugate thereof about once a week for about two to about twelve months. In another embodiment, the invention embraces administration of a polyamine, polyamine analog, non-conformationally restricted polyamine analog, conformationally restricted polyamine analog or conjugate thereof about once every two weeks for about two to about twelve months. In another embodiment, the invention embraces administration of a polyamine, polyamine analog, non-conformationally restricted polyamine analog, conformationally restricted polyamine analog or conjugate thereof about once every three weeks for about two to about twelve months. In another embodiment, the invention embraces administration of a polyamine, polyamine analog, non-conformationally restricted polyamine analog, conformationally restricted polyamine analog or conjugate thereof about once a month for about two to about twelve months. In another embodiment, the invention embraces administration of a polyamine, polyamine analog, non-conformationally restricted polyamine analog, conformationally restricted polyamine analog or conjugate thereof about once every two months for about two to about twelve months. In another embodiment, the invention embraces administration of a polyamine, polyamine analog, non-conformationally restricted polyamine analog, conformationally restricted polyamine analog or conjugate thereof about once every three months for about three to about twelve months. In another embodiment, the invention embraces administration of a polyamine, polyamine analog, non-conformationally restricted polyamine analog, conformationally restricted polyamine analog or conjugate thereof about once every four months for about four to about twelve months. In another embodiment, the invention embraces administration of a polyamine, polyamine analog, non-conformationally restricted polyamine analog, conformationally restricted polyamine analog or conjugate thereof about once every five months for about five to about fifteen months. In another embodiment, the invention embraces administration of a polyamine, polyamine analog, non-conformationally restricted polyamine analog, conformationally restricted polyamine analog or conjugate thereof about once every six months for about six to about twelve months. In another embodiment, the invention embraces administration of a polyamine, polyamine analog, non-conformationally restricted polyamine analog, conformationally restricted polyamine analog or conjugate thereof about once a week, about once every two weeks, about once every three weeks, about once a month, about once every two months, about once every three months, about once every four months, about once every five months, about or once every six months, for an indefinite period of time, or until particular clinical endpoints are met. In another embodiment, the aforementioned administration regimens comprise periadventitial administration of the polyamine, polyamine analog, non-conformationally restricted polyamine analog, conformationally restricted polyamine analog or conjugate thereof. In another embodiment, the aforementioned administration regimens comprise periadventitial injection of the polyamine, polyamine analog, non-conformationally restricted polyamine analog, conformationally restricted polyamine analog or conjugate thereof. In another embodiment, the aforementioned administration regimens comprise administration of CGC-11047. In another embodiment, the aforementioned administration regimens comprise administration of CGC-11093. In another embodiment, the aforementioned administration regimens comprise administration of CGC-11144. In another embodiment, the aforementioned administration regimens comprise administration of CGC-11150. In another embodiment, the aforementioned administration regimens comprise administration of CGC-11157. In another embodiment, the aforementioned administration regimens comprise administration of CGC-11158. In another embodiment, the aforementioned administration regimens comprise administration of CGC-11217. In another embodiment, the aforementioned administration regimens comprise administration of CGC-11237. In another embodiment, the aforementioned administration regimens comprise administration of CGC-11159. In another embodiment, the aforementioned administration regimens comprise administration of CGC-11302.

In one embodiment of the invention, the dosages may be administered in a sustained release formulation or a sustained release implant, such as in an implant which gradually releases the compounds for use in the invention over a period of time, and which allow for the drug to be administered less frequently, such as about once a month, about once every 2-6 months, about once every year, or even a single administration which need not be repeated. The sustained release implants, devices or formulations (such as pellets, microspheres, and the like) can be administered by placement external to a blood vessel or by placement in or near the tissue of the tunica adventitia (periadventitial placement), by any manner known in the art, such as placement during surgery or placement using a catheter.

The compounds for use in the invention can be administered as the sole active ingredient, or can be administered in combination with another active ingredient, including, but not limited to, taxol, microtubule inhibitors, low molecular weight heparins, estradiols, and steroids.

Kits

The invention also provides articles of manufacture and kits containing materials useful for treating and/or preventing vascular hyperplasia. The article of manufacture comprises a container with a label. Suitable containers include, for example, bottles, vials, and test tubes. The containers may be formed from a variety of materials such as glass or plastic. The container holds a composition having an active agent which is effective for treating and/or preventing vascular hyperplasia The active agent in the composition is one or more polyamines, polyamine analogs, non-conformationally restricted polyamine analogs, or conformationally restricted polyamine analogs, or a conjugate thereof, for example, one or more of the conformationally restricted polyamine analogs or conjugates thereof disclosed herein or incorporated by reference herein. The label on the container indicates that the composition is used for treating and/or preventing vascular hyperplasia, and may also indicate directions for use. The composition present in the container can comprise any of the formulations or compositions described herein.

The invention also provides kits comprising any one or more of a polyamine, polyamine analog, non-conformationally restricted polyamine analog, or conformationally restricted polyamine analog or conjugate thereof. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (methods for treating and/or preventing vascular hyperplasia). The composition present in the kit can comprise any of the formulations or compositions described herein.

In other aspects, the kits may be used for any of the methods described herein, including, for example, to treat a patient or subject suffering from or at risk of vascular hyperplasia. The kits may include instructions for practicing any of the methods described herein.

### EXAMPLES

The invention will be further understood by the following examples, which are not intended to limit the invention in any manner.

### Example 1

### Histopathology of Percutaneous Transluminal Angioplasty (PTA)-Injured Porcine Arteriovenous (AV) Grafts with Perivascular Drug Infusion

This example was performed to determine the effect of injections of CGC-11093 and CGC-11159 on the vasculature of pigs following arteriovenous grafting. The histopathology of reactive and inflammatory responses of pig femoral AV grafts 14 days after Mercator Microsyringe infusion with 1 mg CGC-11093 (6 sites), 10 mg CGC-11093 (2 sites), 10 mg CGC-11159 (8 sites), or placebo saline injection (8 sites) in conjunction with percutaneous transluminal angioplasty (PTA) of the graft vein anastomosis and the proximal vein was assessed.

The methods used were as follows: all graft-vein anastomosis/proximal vein (GVA/PV) sites were subjected to angioplasty and perivascular injection via Mercator Microsyringe. Angioplasty was performed and drugs were infused 14 days after graft implant; animals were sacrificed 14 days later for histopathological examination. A total of 24 GVA/PV sites were evaluated. Six sites received 1 mg of CGC-11093; two sites received 10 mg CGC-11093, eight sites received 10 mg of CGC-11159, and eight sites received placebo injection with saline. All injections were performed in conjunction with percutaneous transluminal angioplasty (PTA) of the GVA and PV.

After 14 days, the graft vein anastomosis sites (GVA/PV) were excised en bloc and perfused with formalin. Sectioning of the graft vein anastomosis was performed as follows: briefly, tissue sections were taken at approximately 5 mm intervals through the proximal vein (PV). Tissue sections were taken at approximately 3 mm intervals through the graft-vein anastomosis (GVA) site. Sections were taken at approximately 5 cm intervals through the distal vein (DV). All tissues were routinely processed, embedded in paraffin, sectioned at 5 microns and stained with hematoxylin and eosin for histopathologic evaluation. Numerous histologic sections through each PV, DV and GVA were evaluated. Each section was labeled with the letter P, G, or D (P=proximal vein, G=graft vein anastomosis, and D=Distal vein) and with a corresponding number to indicate proximity to the graft vein anastomosis.

The histopathologic evaluation included a general description of relevant features regardirig tissue reaction and inflammation around the graft vein anastomosis (GVA) and through the proximal femoral vein (PV). Histopathologic features including inflammation, fibrosis, angiogenesis and venous neointimal hyperplasia (VNH) within the GVA and PV were assessed. Attempts were made to differentiate between histopathologic changes attributed to the surgical procedure, secondary affects of GVA, angioplasty and perivascular injection of drug via Mercator Microsyringe.

Initial histopathologic evaluation was performed on all tissues to define the common inflammatory and reactive changes in all specimens. An overall general description of changes in sections of PV and GVA was made. An estimate of the percent occlusion of the most severely affected segment in both the PV and GVA was made. Two precise, perpendicular cross sectional measurements of the lumen were made in the most severely affected section of the PV. In the GVA a single luminal diameter was measured in the most severely affected section. Next, a qualitative assessment of overall severity of VNH, as well as inflammation within the VNH and degree of angiogenesis within the VNH was made for both the PV and the GVA. Additionally, a general description of the overall perivascular tissue reaction around the PV and the GVA was recorded. Lastly, unique, and potentially significant, pathologic features were documented. Unique pathologic features generally included interpretation of the overall severity of the changes, presence or absence of an inflamed seroma, or presence of thrombosis or infection in the GVA or PV. When assessing the degree or severity of a certain histopathologic change (i.e. severity of VNH, etc) a qualitative scoring method was used. A score of 0=absent, 1=minimal, 2=mild, 3=moderate, 4=severe.

All sections of the GVA/PV exhibit consistent tissue reaction and inflammatory changes that are interpreted as a common reaction resulting from surgical manipulation associated with the graft-vein anastomosis procedure, including implantation of surgical foreign material (graft and suture material), as well as subsequent post-surgical healing (scar formation). Perivascular pathology was mostly consistent throughout all individual samples and was characterized by organizing fibroplasia, mild to moderate (predominately mononuclear) inflammation, and multifocal foreign body granulomas, often centered on suture material or the graft itself. There was also mild to moderate edema, modest numbers of hemosiderin laden macrophages, variable acute hemorrhages, and consistent neovascularization within the fibrous connective tissue scar. Perivascular fibrosis and inflammation typically filled the area between the two arms of the graft and dissected into the surrounding connective tissue and skeletal muscle. Occasionally, muscle bundles were entrapped by fibrosis and there was minimal to mild amounts of skeletal muscle degeneration and regeneration. In most sections, the graft itself elicited a consistent, but mild, foreign body inflammatory response characterized by lamellar fibrosis, patchy lymphoplasmacytic inflammation and occasional macrophages and multinucleated giant cells distributed around the edge of the graft. Often, large reactive macrophages, multinucleated giant cells, lymphocytes and eosinophils surrounded embedded suture material throughout the sections (foreign body reaction).

Several exceptions to the “normal” perivascular tissue reaction were seen. In animals treated with 10 mg CGC-11159, there was often prominent, nodular lymphofollicular inflammation forming a rim surrounding the outside surface of the graft. This prominent lymphofollicular inflammation was not observed in animals treated with other drugs.

Additionally, many of the animals developed graft/vein thrombosis (sterile). Graft/vein thrombosis was associated with variations from the “typical” histopathologic findings both within the lumen of the vessel and, more subtly, in the perivascular tissues. Sterile thrombi in the GVA varied in character. Some thrombi appeared more acute and were characterized by an occlusive fibrin meshwork and entrapped red blood cells. Other thrombi were more organized and characterized by more dense reactive fibrosis and recanalization. Animals treated with 10 mg CGC-11159 developed fibrin thrombosis within the graft, with some degree of resultant vascular and/or graft occlusion, in five of eight sites treated.

Animals treated with placebo saline injection developed a broad spectrum of histologic changes that were also seen to varying degrees in other animals treated with different pharmacotherapy.

In several of the animals, the perivascular adventitia was variably distorted and expanded by a lake of a flocculent, brightly eosinophilic, amorphous to slightly lamellar, acellular matrix. Karyorrhectic debris, acute hemorrhage and degenerate red blood cells were frequently admixed. The inflammatory reaction around this material was variable. Often there was a peripheral rim of multinucleated giant cells, macrophages, lymphocytes and plasma cells with variable fibroplasia. This flocculent eosinophilic material and tissue reaction was often located between the two arms of the graft and/or around the graft vein anastomosis site. This material and tissue reaction is referred to as “inflamed seroma” through the rest of the report. The definitive nature of this material is undetermined.

The following tables highlight some relevant histopathologic features in each GVA/PV. Table 1 shows the estimated percentage of luminal occlusion in the most severely affected portion of both the PV and GVA. The most severely affected PV/GVA are listed in descending order from the top. Most animals that received 10 mg of CGC-11159 developed more severe luminal occlusion or stenosis of the PV and GVA (generally associated with thrombosis of the graft). Also notice that animals that received 1 mg of CGC-11093 generally exhibited less severe vascular occlusion than other animals in the study. Placebo injections are widely distributed.

TABLE 1
Sorted by % Occlusion of PV and % Occlusion of GVA

| | | % | % |
|---|---|---|---|
| Animal | | Occlusion | Occlusion |
| ID | Injectate | of PV | of GVA |
| 6094-2 | 10 mg, CGC-11159 | 100 | 100 |
| 6095-1 | 10 mg, CGC-11159 | 100 | 100 |
| 6038-1 | Placebo | 95 | 100 |
| 6099-1 | 10 mg, CGC-11159 | 80 | 90 |
| 6056-2 | 1 mg, CGC-11093 | 70 | 40 |
| 6099-2 | 10 mg, CGC-11159 | 50 | 25 |
| 6054-2 | Placebo | 40 | 40 |
| 6058-2 | 10 mg, CGC-11093 | 35 | 10 |
| 6055-2 | Placebo | 30 | 15 |
| 6038-2 | Placebo | 25 | 15 |
| 6095-2 | 10 mg, CGC-11159 | 20 | 30 |
| 6098-1 | 10 mg, CGC-11159 | 15 | 15 |
| 6072-1 | 1 mg, CGC-11093 | 15 | 3 |
| 6056-1 | 1 mg, CGC-11093 | 15 | 2 |
| 6072-2 | 1 mg, CGC-11093 | 10 | 45 |
| 6054-1 | Placebo | 10 | 20 |
| 6058-1 | 10 mg, CGC-11093 | 10 | 10 |
| 6094-1 | 10 mg, CGC-11159 | 7 | 45 |
| 6052-1 | Placebo | 7 | 3 |
| 6052-2 | Placebo | 5 | 40 |
| 6098-2 | 10 mg, CGC-11159 | 5 | 7 |
| 6057-2 | 1 mg, CGC-11093 | 5 | 1 |
| 6057-1 | 1 mg, CGC-11093 | 2 | 2 |
| 6055-1 | Placebo | n/a | 10 |

Table 2 evaluates the luminal measurement from the most severely affected section of the PV. Animals with a more widely patent PV are listed near the top while animals that exhibited complete thrombosis or occlusion are listed near the bottom. Notice animals treated with 10 mg of CGC-11159 were more likely to have a smaller luminal diameter.

TABLE 2
Sorted by Luminal Measurement

| | | Luminal |
|---|---|---|
| Animal | | Meas. |
| ID | Injectate | of PV |
| 6095-2 | 10 mg, CGC-11159 | 7.5 × 4 |
| 6057-1 | 1 mg, CGC-11093 | 7 × 6 |
| 6057-2 | 1 mg, CGC-11093 | 6.5 × 6 |
| 6058-1 | 10 mg, CGC-11093 | 6.5 × 6 |
| 6056-1 | 1 mg, CGC-11093 | 6.5 × 3.25 |
| 6098-2 | 10 mg, CGC-11159 | 6 × 5 |
| 6072-2 | 1 mg, CGC-11093 | 6 × 1.7 |
| 6038-2 | Placebo | 5.5 × 3.5 |
| 6054-1 | Placebo | 5 × 3.5 |
| 6098-1 | 10 mg, CGC-11159 | 5 × 3.25 |
| 6072-1 | 1 mg, CGC-11093 | 4.7 × 3.5 |
| 6052-2 | Placebo | 4.5 × 3.75 |
| 6099-2 | 10 mg, CGC-11159 | 4 × 3.5 |
| 6055-2 | Placebo | 4 × 2.5 |
| 6052-1 | Placebo | 3.75 × 3.2 |
| 6058-2 | 10 mg, CGC-11093 | 3.5 × 3.5 |
| 6054-2 | Placebo | 3.5 × 2.25 |
| 6094-1 | 10 mg, CGC-11159 | 2.5 × 3.25 |
| 6099-1 | 10 mg, CGC-11159 | 2 × 1.5 |
| 6038-1 | Placebo | 2 × 0.25 |
| 6056-2 | 1 mg, CGC-11093 | 1.5 × 2 |
| 6094-2 | 10 mg, CGC-11159 | 0 |
| 6095-1 | 10 mg, CGC-11159 | 0 |
| 6055-1 | Placebo | n/a |

Table 3 lists all animals by their number I.D. and provides succinct information on potentially significant unique histopathologic features. Notice that animals that received 10 mg of CGC-11159 often developed luminal thrombosis of both the PV and GVA as well as prominent lymphofollicular inflammation surrounding the graft material. Also notice animals that received 1 mg of CGC-11093 generally had fewer significant or potentially deleterious histopathologic features.

TABLE 3
Sorted by Injectate to show unique features

| Animal | | |
|---|---|---|
| ID | Injectate | Unique Features |
| 6057-1 | 1 mg, CGC-11093 | None |
| 6057-2 | 1 mg, CGC-11093 | Minimal changes |
| 6056-1 | 1 mg, CGC-11093 | None |
| 6072-1 | 1 mg, CGC-11093 | None |
| 6072-2 | 1 mg, CGC-11093 | None |
| 6056-2 | 1 mg, CGC-11093 | Artery distal to the graft appears |
| | | chronically thrombosed and degenerate |
| 6058-1 | 10 mg, CGC-11093 | Mildly affected |
| 6058-2 | 10 mg, CGC-11093 | None |
| 6095-2 | 10 mg, CGC-11159 | Luminal thrombosis and occlusion of the |
| | | graft without obstruction of the vein |
| 6098-2 | 10 mg, CGC-11159 | Lymphofollicular inflammation |
| | | surrounding graft |
| 6099-2 | 10 mg, CGC-11159 | Acute fibrin thrombosis of the graft |
| | | and prominent lymphofollicular |
| | | inflammation around the graft |
| 6098-1 | 10 mg, CGC-11159 | Lymphofollicular inflammation |
| | | surrounding graft |
| 6094-1 | 10 mg, CGC-11159 | None |
| 6099-1 | 10 mg, CGC 11159 | Organizing luminal thrombosis in the |
| | | graft |
| 6094-2 | 10 mg, CGC-11159 | Chronic thrombosis of GVA, well |
| | | organized with recanalization |
| 6095-1 | 10 mg, CGC-11159 | Luminal thrombosis and occlusion of |
| | | GVA |
| 6055-1 | Placebo | None |
| 6054-1 | Placebo | None |
| 6038-2 | Placebo | Small inflamed seroma |
| 6055-2 | Placebo | Perivascular inflamed seroma |
| 6052-2 | Placebo | None |
| 6052-1 | Placebo | Minimally affected specimen |
| 6054-2 | Placebo | Prominent luminal fibrin and nodular |
| | | VNH at the toe of the graft |
| 6038-1 | Placebo | Marked luminal stenosis with acute |
| | | fibrin occlusion, perivascular |
| | | inflamed seroma |

Table 4 lists the qualitative assessment for severity of overall VNH in both the PV and GVA. More severely affected samples are listed in descending order from the top. Notice animals treated with 10 mg of CGC-11159 seem to have developed more severe VNH in both the PV and GVA. Also notice animals that received 1 mg of CGC-11093 generally exhibit a minimal amount of VNH in both the PV and GVA.

TABLE 4
Sorted by Severity of VNH in PV and GVA

| Animal | | Severity of | Severity of |
|---|---|---|---|
| ID | Injectate | VNH in PV | VNH in GVA |
| 6099-1 | 10 mg, CGC-11159 | 4 | 4 |
| 6094-2 | 10 mg, CGC-11159 | 4 | 4 |
| 6095-1 | 10 mg, CGC-11159 | 4 | 4 |
| 6038-1 | Placebo | 4 | 4 |
| 6099-2 | 10 mg, CGC-11159 | 4 | 3 |
| 6054-2 | Placebo | 3 | 3 |
| 6056-2 | 1 mg, CGC-11093 | 3 | 2 |
| 6058-2 | 10 mg, CGC-11093 | 3 | 2 |
| 6072-2 | 1 mg, CGC-11093 | 2 | 3 |
| 6094-1 | 10 mg, CGC-11159 | 2 | 3 |
| 6038-2 | Placebo | 2 | 2 |
| 6056-1 | 1 mg, CGC-11093 | 2 | 1 |
| 6072-1 | 1 mg, CGC-11093 | 2 | 1 |
| 6055-2 | Placebo | 2 | 1 |
| 6095-2 | 10 mg, CGC-11159 | 1 | 3 |
| 6098-2 | 10 mg, CGC-11159 | 1 | 2 |
| 6098-1 | 10 mg, CGC-11159 | 1 | 2 |
| 6054-1 | Placebo | 1 | 2 |
| 6052-2 | Placebo | 1 | 2 |
| 6057-1 | 1 mg, CGC-11093 | 1 | 1 |
| 6057-2 | 1 mg, CGC-11093 | 1 | 1 |
| 6058-1 | 10 mg, CGC-11093 | 1 | 1 |
| 6052-1 | Placebo | 1 | 1 |
| 6055-1 | Placebo | n/a | 2 |

0 = absent, 1 = minimal, 2 = mild, 3 = moderate, 4 = severe.

Table 5 lists the qualitative score for severity of VNH inflammation in both the PV and GVA. More severely affected sections are listed in descending order from the top. Notice animals treated with 10 mg of CGC-11159 generally exhibited a greater severity of VNH inflammation (probably associated with thrombus formation) while animals that received 1 mg of CGC-11093 generally exhibited minimal VNH inflammation.

TABLE 5
Sorted by Severity of VNH Inflammation in PV and in GVA

| | | Severity of VNH | Severity of VNH |
|---|---|---|---|
| Animal | | Inflammation | Inflammation |
| ID | Injectate | in PV | in GVA |
| 6099-1 | 10 mg, CGC-11159 | 4 | 4 |
| 6038-1 | Placebo | 4 | 3 |
| 6095-1 | 10 mg, CGC-11159 | 3 | 3 |
| 6094-2 | 10 mg, CGC-11159 | 2 | 4 |
| 6099-2 | 10 mg, CGC-11159 | 2 | 2 |
| 6054-2 | Placebo | 2 | 2 |
| 6098-1 | 10 mg, CGC-11159 | 2 | 2 |
| 6058-2 | 10 mg, CGC-11093 | 1 | 2 |
| 6072-2 | 1 mg, CGC-11093 | 1 | 2 |
| 6095-2 | 10 mg, CGC-11159 | 1 | 2 |
| 6056-2 | 1 mg, CGC-11093 | 1 | 1 |
| 6094-1 | 10 mg, CGC-11159 | 1 | 1 |
| 6038-2 | Placebo | 1 | 1 |
| 6056-1 | 1 mg, CGC-11093 | 1 | 1 |
| 6072-1 | 1 mg, CGC-11093 | 1 | 1 |
| 6055-2 | Placebo | 1 | 1 |
| 6098-2 | 10 mg, CGC-11159 | 1 | 1 |
| 6057-1 | 1 mg, CGC-11093 | 1 | 1 |
| 6058-1 | 10 mg, CGC-11093 | 1 | 1 |
| 6057-2 | 1 mg, CGC-11093 | 1 | 0 |
| 6052-1 | Placebo | 1 | 0 |
| 6054-1 | Placebo | 0 | 2 |
| 6052-2 | Placebo | 0 | 2 |
| 6055-1 | Placebo | n/a | 1 |

0 = absent, 1 = minimal, 2 = mild, 3 = moderate, 4 = severe.

Table 6 lists the qualitative score for degree of VNH angiogenesis in both the PV and GVA. More severely affected segments are listed in descending order from the top. Notice animals treated with 10 mg of CGC-11159 generally exhibited a greater degree of VNH angiogenesis in the PV and GVA while animals treated with 1 mg of CGC-11093 generally developed minimal to absent VNH angiogenesis.

TABLE 6
Sorted by Degree of VNH Angiogenesis in PV and in GVA

| | | Severity of VNH | Severity of VNH |
|---|---|---|---|
| Animal | | Angiogenesis | Angiogenesis |
| ID | Injectate | in PV | in GVA |
| 6099-1 | 10 mg, CGC-11159 | 4 | 4 |
| 6038-1 | Placebo | 4 | 3 |
| 6094-2 | 10 mg, CGC-11159 | 3 | 4 |
| 6095-1 | 10 mg, CGC-11159 | 3 | 3 |
| 6099-2 | 10 mg, CGC-11159 | 3 | 1 |
| 6095-2 | 10 mg, CGC-11159 | 2 | 3 |
| 6054-2 | Placebo | 2 | 1 |
| 6058-2 | 10 mg, CGC-11093 | 1 | 1 |
| 6072-2 | 1 mg, CGC-11093 | 1 | 1 |
| 6056-2 | 1 mg, CGC-11093 | 1 | 1 |
| 6094-1 | 10 mg, CGC-11159 | 1 | 1 |
| 6038-2 | Placebo | 1 | 1 |
| 6055-2 | Placebo | 1 | 1 |
| 6054-1 | Placebo | 1 | 1 |
| 6072-1 | 1 mg, CGC-11093 | 1 | 0 |
| 6058-1 | 10 mg, CGC-11093 | 1 | 0 |
| 6052-2 | Placebo | 0 | 2 |
| 6098-1 | 10 mg, CGC-11159 | 0 | 1 |
| 6098-2 | 10 mg, CGC-11159 | 0 | 1 |
| 6057-1 | 1 mg, CGC-11093 | 0 | 1 |
| 6056-1 | 1 mg, CGC-11093 | 0 | 0 |
| 6057-2 | 1 mg, CGC-11093 | 0 | 0 |
| 6052-1 | Placebo | 0 | 0 |
| 6055-1 | Placebo | n/a | 0 |

0 = absent, 1 = minimal, 2 = mild, 3 = moderate, 4 = severe.

Each of the GVA/PV specimens features complex histopathologic changes in response to multiple variables. First, there are histopathologic changes associated with the surgery itself. Surgical manipulation of soft tissues (including arteriotomy, venotomy and graft anastomosis and implantation of foreign material) is traumatic in nature. This type of surgery routinely induces foreign body tissue reaction and inflammation with subsequent tissue damage resolved by fibrosis or scar formation, as seen in the current study. Secondly, exposing the femoral vein to the high blood pressure of the femoral artery (via GVA) induces aggressive venous neointimal hyperplasia (VNH) as previously described (Kidney International, Vol. 62, 2002, pp. 2272-2280). The goal of the femoral graft-vein anastomosis is to induce VNH and thus establish an animal model that can be used to investigate preventive therapies. Aggressive VNH may cause occlusion of either the graft or the proximal vein. The animals in this study were also exposed to percutaneous transluminal angioplasty (PTA) of the graft-vein anastomosis and proximal vein. The histopathologic changes associated with PTA of the GVA are not well documented. In the literature, angioplasty is known to elicit vascular inflammation and neointimal hyperplasia. Both of these histopathologic features were identified in the current study but could not be differentiated from inflammatory and neointimal changes that may have been caused by graft-vein anastomosis alone or in conjunction with surgical manipulation. In addition, these animals were also subjected to perivascular injection of either CGC-11093 or CGC-11159 at different concentrations via Mercator Microsyringe. Differentiating between the pathologic changes attributed to perivascular injection versus the pathologic changes associated with angioplasty, graft-vein anastomosis and surgical manipulation is difficult, if not impossible in the current study. However, unique features were appreciated in animals treated with 10 mg CGC-11159. First, these animals exhibited prominent, nodular to coalescing lymphocytic inflammation around the implanted graft material. This change had not been seen previously in any of the other animals. The drug delivered at this dose may have been at least partially responsible for this unique inflammatory change. In addition, animals treated with 10 mg CGC-11159 seem to have a higher incidence of GVA/PV thrombosis. In contrast, animals treated with 1 mg CGC-11093 generally developed less severe histopathologic changes. In general, most other animals, including saline injected placebos developed histopathologic changes that were similar in nature and severity. These changes were not readily distinguishable from each other in this study.

An unexplained finding in several of these animals was the presence of an inflamed perivascular seroma. The nature of the seroma was not definitely determined. Special histochemical stains applied to the inflamed seroma indicate that it is not derived purely from red blood cells, nor is it derived purely from fibrin. The histomorphologic features of the material suggests it is comprised of fluid. The duration that this material has been present within the perivascular tissues is also not definitively determined. However, the presence of multinucleated giant cells and mild fibroplasia indicate that the material has been present in the perivascular space for a minimum of three to five days. Several potential causes for this inflamed seroma have been considered. (1) It may be associated with perivascular injection via Mercator Microsyringe. However, the material is generally not located at the exact target site of the perivascular injection (as determined via fluoroscopy during perivascular injection). Another potential cause for the inflamed seroma is (2) trauma, or, (3) potential breakdown of the graft vein anastomosis with leakage of plasma into the perivascular tissues. Trauma may have occurred during the angioplasty procedure or for other unknown reasons during the postoperative period.

To summarize the results, a 49.2% decrease in luminal stenosis versus placebo was observed with CGC-11093 injections. A 22.6% decrease in luminal stenosis versus placebo was observed with CGC-11159, although more prominent abnormal perivascular tissue reactions were observed at this dose of CGC-11159. Thus the polyamine analogs were able to decrease the blockage of the vessel in comparison to placebo.

### Example 2

### Inhibition of Graft Intimal Hyperplasia by Polyamine Infusion

This example demonstrates the results of polyamine administration in a baboon model of bypass grafts. The procedure of Chen et al., Journal of Vascular Surgery 31(2):354 (2000) was used to prepare the animals. Briefly, baboons underwent bilateral aortoiliac bypass grafting with expanded polytetrafluoroethylene grafts. The distal anastomosis of one graft (the left graft) was infused with polyamine; the distal anastomosis of the contralateral graft (right graft) was infused with placebo (saline). The drugs were infused with the Alzet 2ML4 pump at a flow rate of 2.5 ul/hr for 28 days. For administration of CGC-11093, the pump contained 4.4 mg/ml (10 mM) drug and delivered 266 ug/day (7.5 mg in 28 days). For administration of CGC-11159, drug concentration was also at 10 mM; the pump delivered 646.2 ug/day (18.1 mg in 28 days). After 28 days the grafted vessels were harvested and analyzed. All anastomosis/graft samples were harvested by in vivo pressure-perfusion fixation with 10% Neutral Buffered Formalin (NBF) and fixed further overnight in 10% NBF. Samples were cut into several sections (noting proximal and distal) and marked with green dye for standard overnight processing. Processed samples were embedded in paraffin sectioned at 4 mm intervals and stained with conventional hematoxylin and eosin (H&E). For morphometric measurements and analysis, H&E sections were imaged with a Nikon EFD-3 microscope with darkfield and polarized light epiluminescence and an RT Slider Diagnostic Camera. Graft and vessel sections were analyzed with respect to total neointimal area and maximal thickness. Morphometric analysis was performed using Image-Pro Plus 4.5 (Media Cybernetics) software. All images were digitized and archived.

Of the polyamines tested, treatment of baboon vascular grafts with CGC-11159 showed approximately a 50% decrease in average maximal thickness of hyperplasia in the vascular graft (left distal anastomosis) as compared with placebo-treated grafts (right proximal and distal anastomoses). Note that as flow is unidirectional in the grafts, only the left distal anastomosis would be expected to be exposed to any significant amount of polyamine, as the left proximal anastomosis is upstream of polyamine administration; see Markou et al., Ann. Biomed. Eng. 26:502 (1998) for theoretical and in vitro experimental descriptions of concentrations of infusate.

TABLE 7
thickness of grafts after polyamine or placebo treatment

| | | | | | Left Distal |
|---|---|---|---|---|---|
| | | Right | Left | Right | Ave. |
| | Polyamine | Proximal | Proximal | Distal | (Treated |
| Animal ID | Analog Used | Ave. | Ave. | Ave. | site) |
| 24566 | CGC-11159 | 0.294 | 0.695 | 0.674 | 0.336 |
| 25501 | CGC-11302 | 0.727 | 1.047 | 1.292 | 0.822 |
| 24569 | CGC-11098 | 0.526 | 0.938 | 1.431 | 1.778 |
| 24813 | CGC-11093 | — | 0.499 | — | 1.072 |
| 24815 | CGC-11144 | 0.311 | — | 0.547 | — |

### Example 3

### N-{2-[(4-Ethylamino-butylamino)-methyl]-cyclopropylmethyl}-N′-pentyl-butane-1,4-diamine (CGC-11302)

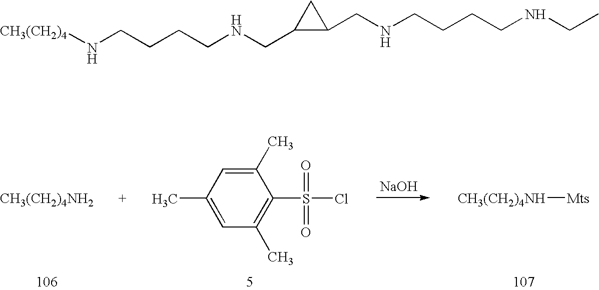

Pentylamine (106, 2.0 g, 22.9 mmol) in 30 ml CHCl₃ was mixed at 0° C. with 27 ml 2N NaOH. 2-mesitylenesulfonyl chloride (5, 4.99 g, 22.9 mmol) was added and the reaction mixture was stirred for 2 hrs at room temperature. The reaction was monitored by TLC (silica, hexane:EtOAc 7:3). The CHCl₃ phase was separated, washed with NH₄Cl (2×), dried, and evaporated to provide 6.16 g (100%) of N-pentyl-mesitylene-2-sulfonamide (107). ¹³C and ¹H NMR confirmed the identity of the product.

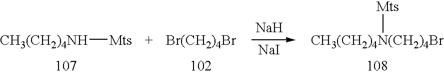

N-pentyl-mesitylene-2-sulfonamide (107, 6.16 g, 22.8 mmol) and 1,4-dibromobutane (102, 49.2 g, 27 ml, 228 mmol) were dissolved in 60 ml DMF and cooled to 0° C. 1.0 g of a 60% NaH suspension in oil (27.36 mmol) was added and the mixture stirred at room temperature for 20 minutes. NaI (3.4 g, 22.8 mmol) was added and the reaction mixture heated to 75° C. for 2 hours. The reaction was monitored by TLC (silica, hexane:EtOAc 9:1). The DMF was evaporated off, and the residue was dissolved in CHCl₃, washed with NH₄Cl solution (2×), dried, evaporated, and the product was purified by silica gel column (hexane:EtOAc 9.5:0.5) to provide N-4-bromobutyl-N-pentyl-mesitylene-2-sulfonamide (108, 7.27 g, 79%).

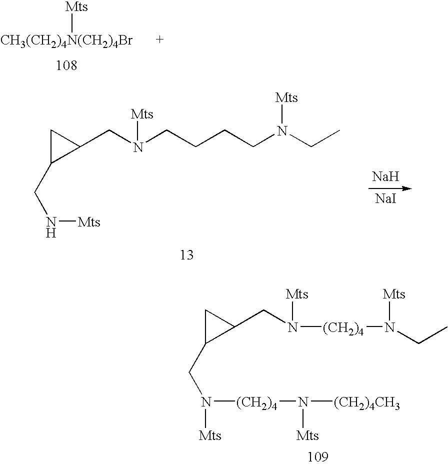

N-4-bromobutyl-N-pentyl-mesitylene-2-sulfonamide (108, 808 mg, 2.0 mmol) and N-(2-mesitylene-2-sulfonamidomethylcyclopropylmethyl)-N-mesitylene-2-sulfonyl-N′-mesitylene-2-sulfonyl-N′-ethyl-1,4-diaminobutane (13, racemic mixture of trans isomers, 1.52 g, 2.0 mmol) were dissolved in 25 ml DMF. 96 mg of a 60% NaH suspension in oil (2.4 mmol) was added and the mixture stirred at room temperature for 20 minutes. NaI (450 mg, 3.0 mmol) was added and the reaction mixture heated to 75-80° C. for 2 hours. The reaction was monitored by TLC (silica, hexane:EtOAc 8:2). The DMF was evaporated off, and the residue was dissolved in CHCl₃, washed with NH₄Cl solution (2×), dried, evaporated, and the product was purified by silica gel column (hexane:EtOAc 8:2) to provide N-(4-{[2-({[4-(Ethyl-mesitylene-2-sulfonyl-amino)-butyl]-mesitylene-2-sulfonyl-amino}-methyl)-cyclopropylmethyl]-mesitylene-2-sulfonyl-amino}-butyl)-N-pentyl-mesitylene-2-sulfonamide (109, racemic mixture of trans isomers, 1.9 g, 87%).

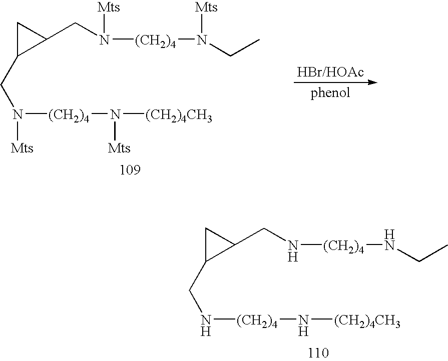

The compound 109 (racemic mixture of trans isomers, 1.9 g, 1.77 mmol) was dissolved in 25 ml CH₂Cl₂ with 25 ml of 30% HBr in HOAc and 4.2 g of phenol, and the mixture reacted for 18 hours. The mixture was cooled, water (50 ml) was added and the aqueous suspension was separated from the organic phase, then washed with 3×20 ml of CH₂Cl₂. The aqueous phase was adjusted with 5 ml of 2N NaOH and 4 ml of 50% KOH and extracted with 3×20 ml of CHCl₃. The CHCl₃ was dried. and evaporated and the product dissolved in ethanol. This was cooled and concentrated HCl was added to the precipitate. The product was filtered to provide 580 mg (65%) of the title compound 110, CGC-11302 (racemic mixture of trans isomers) isolated as the tetrahydrochloride salt.

The disclosures of all publications, patents, patent applications and published patent applications referred to herein by an identifying citation are hereby incorporated herein by reference in their entirety.

Although the foregoing invention has been described in some detail for purposes of clarity of understanding, it is apparent to those skilled in the art that certain minor changes and modifications will be practiced. Therefore, any description or examples should not be construed as limiting the scope of the invention.

What is claimed is:

1. A method of treating or preventing vascular hyperplasia, comprising:
administering one or more polyamines, polyamine analogs, non-conformationally restricted polyamine analogs, or conformationally restricted polyamine analogs, or a conjugate thereof, in an amount effective to treat or prevent vascular hyperplasia, to a subject with vascular hyperplasia or at risk of vascular hyperplasia.

2. The method of claim 1, comprising a method of treating vascular hyperplasia comprising administering one or more polyamines, polyamine analogs, non-conformationally restricted polyamine analogs, or conformationally restricted polyamine analogs, or a conjugate thereof, in an amount effective to treat vascular hyperplasia, to a subject with vascular hyperplasia.

3. The method of claim 1, comprising a method of preventing vascular hyperplasia comprising administering one or more polyamines, polyamine analogs, non-conformationally restricted polyamine analogs, or conformationally restricted polyamine analogs, or a conjugate thereof, in an amount effective to prevent vascular hyperplasia, to a subject at risk of vascular hyperplasia.

4. The method of claim 1, wherein the one or more polyamines, polyamine analogs, non-conformationally restricted polyamine analogs, conformationally restricted polyamine analogs, or conjugate thereof is a conformationally restricted polyamine analog.

5. The method of claim 1, wherein the one or more polyamines, polyamine analogs, non-conformationally restricted polyamine analogs, conformationally restricted polyamine analogs, or conjugate thereof is one or more non-conformationally restricted polyamine analogs.

6. The method of claim 5, wherein the one or more non-conformationally restricted polyamine analogs is selected from the group of compounds of the formula: 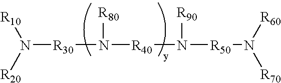
where R₁₀, R₂₀, R₆₀, and R₇₀ are independently selected from H, methyl, ethyl, n-propyl, isopropyl, n-butyl, isobutyl, sec-butyl, and t-butyl;
where each R₈₀ and R₉₀ are independently selected from H, methyl, ethyl, n-propyl, isopropyl, n-butyl, isobutyl, sec-butyl, and t-butyl;
where R₃₀, each R_{40,} and R₅₀ are independently selected from:
—CH₂CH₂CH₂CH₂—
—CHOFICH₂CH₂CH₂—
—CH₂CHOHCH₂CH₂—
—CH₂CH₂CHOHCH₂—
—CH₂CH₂CH₂CHOH—
—CH₂CH₂CH₂—
—CHOHCH₂CH₂—
—CH₂CHOHCH₂—
—CH₂CH₂CHOH;—
and where y is an integer selected from 5, 6, 7, 8, 9, 10, 11, 12, and 13; and all salts thereof.

7. The method of claim 6, wherein the one or more non-conformationally restricted polyamine analogs is selected from the group of compounds of the formula:
CH₃CH₂—NH—(CH₂CH₂CH₂CH₂—NH—)₉—CH₂CH₃,
CH₃CH₂—NH—(CH₂CH₂CH₂CH₂—NH—)₇—CH₂CH₃,
CH₃CH₂—NH—(CH₂CH₂CH₂CH₂—NH—)₁₃—CH₂CH₃, and
CH₃CH₂—NH—(CH₂CH₂CH₂CH₂—NH—)₁₁—CH₂CH₃,
and all salts thereof.

8. The method of claim 7, wherein the one or more non-conformationally restricted polyamine analogs is:
CH₃CH₂—NH—(CH₂CH₂CH₂CH₂—NH—)₉—CH₂CH₃
or a salt thereof.

9. The method of claim 1, wherein the one or more polyamines, polyamine analogs, non-conformationally restricted polyamine analogs, conformationally restricted polyamine analogs, or conjugate thereof is selected from the group of compounds of the formula
E—NH—D—NH—B—A—B—NH—D—NH—E
wherein A is independently selected from the group consisting of C₂-C₆ alkene and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl; and all salts, hydrates, solvates, and stereoisomers thereof.

10. The method of claim 9, wherein the one or more polyamines, polyamine analogs, non-conformationally restricted polyamine analogs, conformationally restricted polyamine analogs, or conjugate thereof is selected from the group consisting of 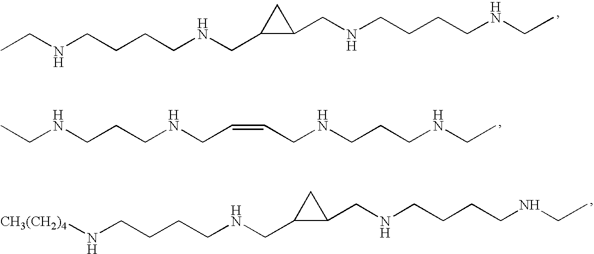
and all stereoisomers, salts, hydrates, and solvates thereof.

11. The method of claim 1, wherein the one or more polyamines, polyamine analogs, non-conformationally restricted polyamine analogs, conformationally restricted polyamine analogs, or conjugate thereof is selected from the group consisting of 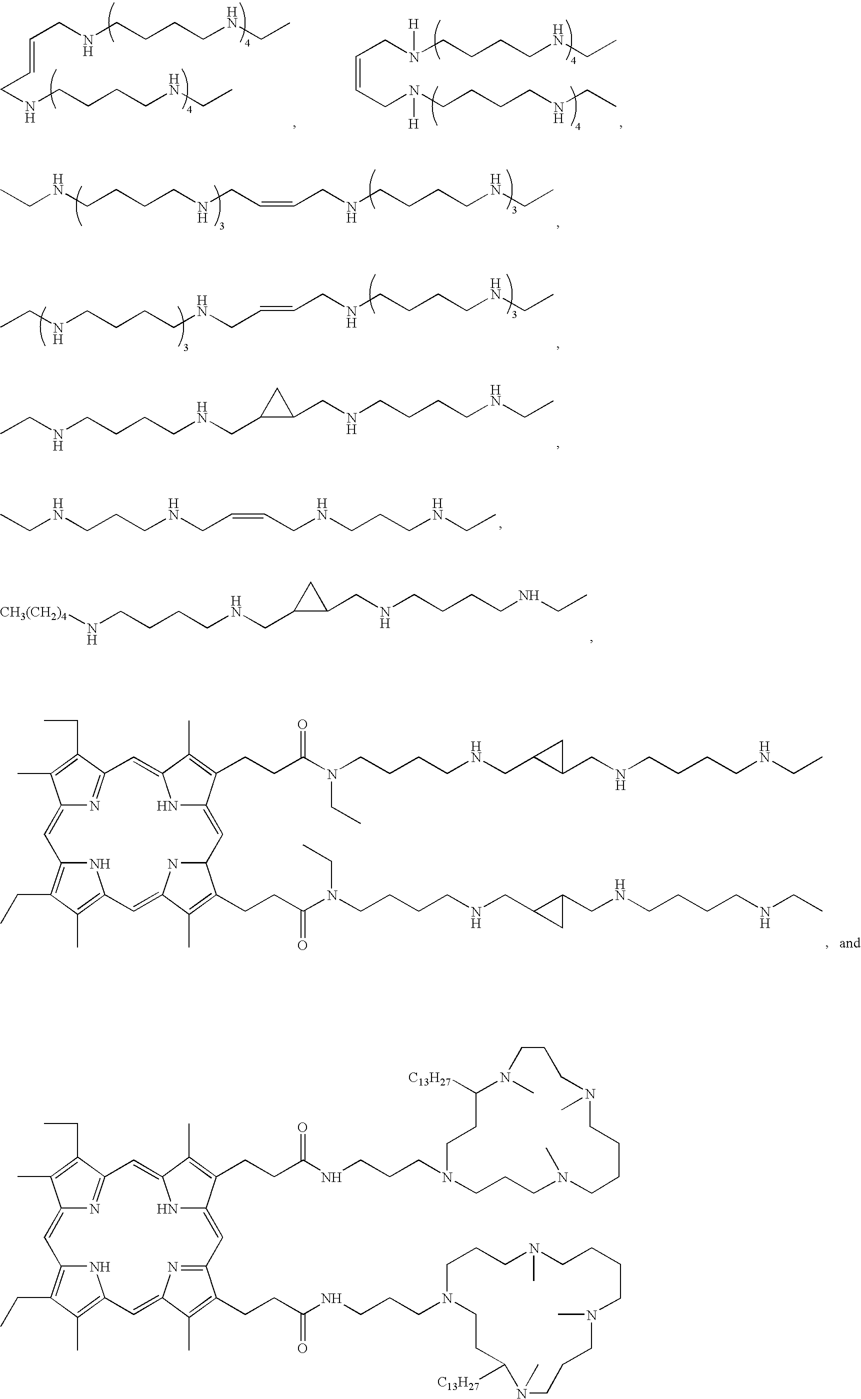
and all stereoisomers, salts, hydrates, and solvates thereof.

12. The method of claim 1, wherein the conjugate of a polyamine, polyamine analog, non-conformationally restricted polyamine analog, or conformationally restricted polyamine analog is a covalent conjugate with a peptide, amino acid, or porphyrin.

13. The method of claim 1, wherein the subject with vascular hyperplasia or at risk of vascular hyperplasia has undergone angioplasty.

14. The method of claim 1, wherein the subject with vascular hyperplasia or at risk of vascular hyperplasia has undergone atherectomy.

15. The method of claim 1, wherein the subject with vascular hyperplasia or at risk of vascular hyperplasia has undergone endarterectomy.

16. The method of claim 1, wherein the subject with vascular hyperplasia or at risk of vascular hyperplasia has undergone stent implantation.

17. The method of claim 1, wherein the subject with vascular hyperplasia or at risk of vascular hyperplasia has received a vascular graft.

18. The method of claim 17, wherein the vascular graft is a blood vessel used in a bypass operation.

19. The method of claim 17, wherein the vascular graft is a device used for vascular access.

20. The method of claim 17, wherein the vascular graft is an arteriovenous fistula used for vascular access.

21. The method of claim 11, wherein the one or more polyamines, polyamine analogs, non-conformationally restricted polyamine analogs, conformationally restricted polyamine analogs, or conjugate thereof is administered in a composition suitable for periadventitial administration, and the composition is administered at or near a site of vascular hyperplasia or at risk of vascular hyperplasia via periadventitial administration.

22. The method of claim 21, wherein the periadventitial administration is periadventitial injection.

23. The method of claim 21, wherein the periadventitial administration is performed at or near a site of vascular hyperplasia or at risk of vascular hyperplasia, during surgery.

24. The method of claim 22, wherein the periadventitial injection is performed at or near a site of vascular hyperplasia or at risk of vascular hyperplasia, during surgery.

25. The method of claim 21, wherein the periadventitial administration is performed at or near a site of vascular hyperplasia or at risk of vascular hyperplasia using a catheter.

26. The method of claim 22, wherein the periadventitial injection is performed at or near a site of vascular hyperplasia or at risk of vascular hyperplasia using a catheter.

27. The method of claim 1, wherein the one or more polyamines, polyamine analogs, non-conformationally restricted polyamine analogs, conformationally restricted polyamine analogs, or conjugate thereof is administered via a sustained release formulation, a sustained release implant, or a sustained release device.

28. The method of claim 1, wherein the one or more polyamines, polyamine analogs, non-conformationally restricted polyamine analogs, conformationally restricted polyamine analogs, or conjugate thereof is administered via a sustained release formulation applied to a stent.

29. The method of claim 1, wherein the one or more polyamines, polyamine analogs, non-conformationally restricted polyamine analogs, conformationally restricted polyamine analogs, or conjugate thereof is administered via a sustained release formulation applied to a vascular graft device.

30. The method of claim 1, wherein the one or more polyamines, polyamine analogs, non-conformationally restricted polyamine analogs, conformationally restricted polyamine analogs, or conjugate thereof is administered at a frequency of about once a week for about two months to about twelve months, about once every two weeks for about two months to about twelve months, about once every three weeks for about two months to about twelve months, or about once every four weeks for about two months to about twelve months.
